(19) United States
(12) Patent Application Publication
(10) Pub. No.: US 2013/0122528 A1
(43) Pub. Date: May 16, 2013

(54) COMPOSITIONS AND METHODS FOR ASSESSING APPENDICITIS

(71) Applicant: AspenBio Pharma, Inc., Castle Rock, CO (US)

(72) Inventors: Steven Patrick TYRELL, Erie, CO (US); Barry Patrick Vant-Hull, Boulder, CO (US); Mark Allen Colgin, Castle Rock, CO (US); Mark Joseph Flipse, Castle Rock, CO (US); Joseph Carey Gogain, Denver, CO (US); Karen Copeland, Boulder, CO (US)

(73) Assignee: AspenBio Pharma, Inc., Castle Rock, CO (US)

(21) Appl. No.: 13/679,965

(22) Filed: Nov. 16, 2012

Related U.S. Application Data
(60) Provisional application No. 61/629,386, filed on Nov. 16, 2011.

(51) Int. Cl. G01N 33/68

(52) U.S. Cl. CPC G01N 33/6893
USPC 435/7.92; 435/287.2; 702/19

(57) ABSTRACT

The invention relates to methods, devices and systems for assessing appendicitis in a subject. More particularly, this invention relates to methods, devices and systems for assessing appendicitis in a subject by evaluating multiple biomarkers in a sample from the subject and comparing the values of the biomarker to a reference value from a group having high or low risk for appendicitis, or combining the values of the biomarkers using a mathematical algorithm to produce a numerical test score, and comparing the test score to a reference value to assess appendicitis in the subject.

### CROSS-REFERENCE TO RELATED APPLICATION

The present application claims the priority benefit of U.S. provisional application Ser. No. 61/629,386, filed Nov. 16, 2011. The content of the U.S. provisional application is incorporated by reference herein in its entirety.

### TECHNICAL FIELD

The invention relates to methods, devices and systems for assessing appendicitis in a subject. More particularly, this invention relates to methods, devices and systems for assessing appendicitis in a subject by evaluating multiple biomarkers in a sample from the subject and comparing the values of the biomarker to a reference value from a group having high or low risk for appendicitis, or combining the values of the biomarkers using a mathematical algorithm to produce a numerical test score, and comparing the test score to a reference value to assess appendicitis in the subject.

### BACKGROUND ART

Approximately 9.5 million patients visit emergency departments (ED) in the United States each year with the primary complaint of abdominal pain (2009 data).¹ Approximately 2 million or 22% of those are children.¹ The evaluation of abdominal pain is complex and requires a broad understanding of all possible mechanisms responsible for pain and recognition of typical patterns and clinical presentations. Because all patients may not have a classic clinical presentation, unusual causes of abdominal pain must also be considered relative to their demographic groups e.g., children, females of child-bearing age, elderly or the immunocompromised patients. A variety of basic laboratory tests and imaging techniques are employed to assist the physician in determining the cause of pain and eventually, and exact diagnosis. Commonly employed tests are shown in Table 1 below.

TABLE 1
Commonly employed tests for abdominal pain

| Diagnostic Tests Performed | | |
|---|---|---|
| for Abdominal Pain | 2009 | |
| CBC (Complete Blood Count) | | |
| All patients | 6.6 | mil |
| Children | 1 | mil |
| CT (Cat Scan) | | |
| All patients | 3.1 | mil |
| Children | 0.41 | mil |
| US (Ultrasound) | | |
| All patients | 1.2 | mil |
| Children | 0.26 | mil |

Acute appendicitis accounts for about 2.4% of all ED visits and is the most common cause of abdominal pain requiring emergency surgical intervention.^{1,6} The incidence of appendicitis is highest among young adults (<30 yrs) and children and is diagnosed in older patients with less frequency. In elderly patients, acute abdominal pain is more likely to be associated with catastrophic illness rarely seen in the younger demographics. Acute appendicitis is a medical emergency that, if not treated promptly, can lead to life-threatening complications like abdominal abscess, peritonitis (infection of the lining that surrounds the abdomen), ruptured appendix, sepsis, and shock.

In children, abdominal pain is cited as the most frequent and common complaint presented for medical attention at outpatient clinics and emergency rooms.⁶ And among the group of children visiting the ED, appendicitis is the most common surgical etiology.⁶ Of the more than 229,000 appendectomies performed annually, approximately 99,000 of these are children.¹ Because causes of abdominal pain in children can range widely from something quite simple to something quite serious, diagnostic evaluation to separate the likely appendicitis requiring appendectomy from the non-surgical complaint, becomes extremely important. Even with the diagnostic tools and capabilities available today, the causes of abdominal pain in children are particularly challenging, specifically in the younger age groups where verbal communication is complex and patients tend to be more uncooperative as they deal with pain. Furthermore, the presence of parents and family also adds a dynamic that often tends to complicate and confuse the situation.

Appendicitis is frequently misdiagnosed as gastroenteritis during a patient's initial ED visit—the disease often presents with varied symptomology that may suggest this and/or other illness. In many patients, the cause of abdominal pain often remains undifferentiated after diagnostic testing, illustrating the difficulty faced by physicians when attempting to diagnose appendicitis. Depending on the studies cited, for this diagnostic dilemma, a normal appendix is removed, for example 13-25% of the time.⁸ In the United States, the rate of misdiagnoses in the ED in general, may be as high as 65%, with 5-40% directly related to appendicitis.^{2,3,4} Excessive workload, crowded and overused EDs are identified reasons and contributing factors in cases of misdiagnosis.^{2,9} The practice of pediatric emergency medicine is fraught with legal risk—the diagnosis of appendicitis is cited as the second most common cause of litigation against ED physicians in pediatric cases.^{5,9,10} Although appendicitis is the most, well-studied, specific cause of abdominal pain, it remains a challenge for diagnosis.⁵

Based on the patient as an individual where age and gender are relevant, a thorough patient history and physical signs and symptoms are recorded. The presence of fever, anorexia, nausea, vomiting, diarrhea, right lower quadrant pain with/without tenderness (RLQ), limping due to RLQ, and other patient-specific signs and symptoms are assessed. Laboratory tests such as a complete blood count (CBC), absolute neutrophil count (ANC), differential cell count, and urinalysis may be initially ordered to determine the presence of infection or other suspect conditions. Often, electrolytes are ordered to determine dehydration as well. Depending on the age and gender of the patient, other laboratory tests may be ordered, for example, in a female patient of child-bearing age, a pregnancy test may be ordered. Dependent on the physician's risk-assessment of the patient's condition, diagnostic imaging in the form of a non-invasive abdominal ultrasound, or a CAT scan (Computed Tomography, CT), with or without contrast, may be ordered in cases where the clinical presentation is equivocal and more information is needed.

In addition to the tangible forms of information and proof provided by the diagnostic tools mentioned above, the physician importantly, utilizes his/her own clinical assessment and judgment to ultimately manage the patient. The hospital may prescribe a standard of care algorithm, similar or the same as one the many clinical scoring systems past published that describe a clinical, standardized pathway for diagnosing the cause for abdominal pain and specifically, appendicitis. The goal and hope is for an improvement of patient outcomes resulting in a reduction in the medicolegal risks and costs to the hospital.

Acute abdominal pain is associated with a myriad of diagnoses, including appendicitis. Various imaging techniques have played important roles in the diagnosis, treatment and management of patients presenting with this emergent condition, though conventional radiography has been surpassed by newer methods and is rarely considered useful except in cases related to bowel obstruction.¹⁷ So it follows that the use of advanced medical imaging techniques (CT or Cat Scan, MRI or Magnetic Resonance Imaging, US or Ultrasonography) for abdominal pain diagnostics have increased dramatically in recent years—the National Center for Health Statistics reported a 122.6% increase in usage from 1999 to 2008. Another study showed a 141% increase in the use of CT for this indication specifically between 1996 and 2005.¹⁷ This is believed to be related to the high degree of accuracy for specific diagnoses like appendicitis, and the rapid patient throughout enabled with the use of the newer multidetector CT scanners.^{6,17}

CT may be performed with/without contrast medium. Contrast medium is believed to improve accuracy and may be administered by one of three methods: orally, rectally or intravenously (IV). All methods possess advantages and disadvantages. IV contrast has the best reported accuracy with the least number of inconclusive scans in most studies.^{5,17} Specifically stated, “IV contrast improved the ability of the radiologist to identify an inflamed appendix.”⁵ The reported disadvantages for use of IV contrast include adverse reactions to contrast medium, invasive procedure that may pose difficulties in children and the elderly, and it cannot be used in patients with imminent renal insufficiency.^{5,17} Oral contrast has similar reported accuracy to IV contrast in some studies and less in others. This method requires time to administer and transit the bowel and is most associated with a marked increase in the time these patients spend in the ED.^{5,17} Rectal contrast is reported to be generally less sensitive than both IV and oral administration routes, takes less time to administer than the latter methods but may be uncomfortable and unpleasant for the patient.^{5,17}

MRI is widely used in the diagnostic work-up of patients presenting with acute abdominal pain. The main MRI advantage is the lack of ionizing radiation exposure—but additionally, high intrinsic contrast resolution is achieved without use of contrast medium. A current associated disadvantage is the availability of technical expertise—MRI is only used in select cases in many institutions. The usefulness of MRI for abdominal pain diagnoses has not yet been established due to relatively limited studies performed, but because it might be a promising alternative to CT, it is being actively studied.¹⁷

Sonography is frequently used in diagnostic work-up of patients with acute abdominal pain, particularly for pediatric patients and women of childbearing age. Related to children, numerous peer-reviewed references discuss the inherent risks and potential outcomes of ionizing radiation exposure for children.^{6,7,16,17} “Children have a higher risk of developing cancer after radiation exposure because they have more years to develop those cancers and are more radiosensitive (children have more actively dividing cells than adults)”⁵. Because of this growing concern, compression sonography has emerged as the first-line tool for evaluating patients, particularly children, with suspected appendicitis. Sonography has many advantages but suffers from some significant disadvantages and therefore may be used to confirm, but not to rule out appendicitis.^{3,5,16} Advantages of sonography is that it is rapid, non-invasive, well-tolerated, relatively inexpensive, radiation-free, sedation-free, and widely available^{5,6,16} Disadvantages of sonography is that it is operator-dependent, requires a high level of skill and expertise, only visualizes a normal appendix in <5% of cases, frequently misses perforated appendices^{5,16,17}

The increased use of CT is of great medical and public concern and the subject of many publications and news worthy broadcasts. It's widespread use has raised medical imaging costs but thought to be cost-effective for cases of appendicitis but not proved in other diagnoses of abdominal pain.¹⁷ For a twenty-five year old, the estimated risk of induced cancer is 1/900 and risk of fatal cancer, 1/1800.¹⁷ For older individuals, the risks are lower. For children, the risks are much higher, and increase as age reduces. When considering CT, the risks must be weighed against the benefits as one in three people will develop cancer in their lifetime.^{6,7,17} The current mainstream diagnostic approach related to the diagnosis of abdominal pain, particularly appendicitis, is a staged imaging approach with US followed by CT, when the US is equivocal and only if necessary and with consideration of patient demographic risks.^{6,7} In the near future, MRI has the potential to advance as a valuable alternative to CT when more data becomes available.^{6,17}

Clinical scoring systems have been developed since the 1980's and best used with the variable array of signs and associated symptoms by helping clinicians deal with the uncertainty provided with current diagnostic techniques. These inexpensive and time-efficient scoring systems have been derived, and occasionally validated, to improve to the identification of patients with disease and improve their outcomes. The first clinical scoring system was developed in 1986 by Alvarado, who reported a clinical scoring tool for AA called the MANTRELS score, reporting a sensitivity of 89%, a specificity of 80% and an overall accuracy of 87%.²⁰ Unfortunately, when Macklin and colleagues applied the MANTRELS criteria to children, the overall sensitivity and specificity fell below 80%.²¹ The following year, a scoring system was prospectively developed by Fenyo in 1987. By evaluating a list of variables, the patient scores were calculated and assigned a number from a baseline value,²² with a reported sensitivity of 86% with specificity of 87%.²³

The first appendicitis scoring system developed specifically in and for children, named the Pediatric Appendicitis Score (PAS), was published by Samuel in 2002 and designed to distinguish children with and without appendicitis using a single discriminate value; it reported a sensitivity of 100% and specificity of 92%,²⁴ yet in two subsequent prospective validation studies, this performance has failed to be reproduced.^{25,26} A second appendicitis score system for children was developed by Lintula and his colleagues in 2005,²⁷ however, it too has not been validated by independent investigators.

It appears the most significant limitations with clinical scoring systems is their attempts to positively predict for the presence of AA, mediocre performance in this usage and aversion of busy clinicians to a ‘checklist’ approach to medicine. Some have suggested the potential for their increased use must be based on a standardized approach to the collection, assimilation, and interpretation of patient information.²⁶

As many patients, particularly children, do not show the classical signs or report the classical symptoms of appendicitis when experiencing abdominal pain, diagnostic tools are welcomed to gain better accuracy.²⁸ Previous studies have shown that WBC counts, C-reactive protein (CRP) and Interleukin-6 can be helpful tools to support clinical diagnosis of appendicitis in children. However, it has also been proposed that these tests could be used to identify patients at low risk of appendicitis.

Appendicitis tests that are easy to use and reduce or avoid disadvantages of the current diagnostic tests for appendicitis, e.g., the various imaging techniques (CT or CAT Scan, MRI or Magnetic Resonance Imaging, US or Ultrasonography), are needed. The present invention addresses this and other related needs in the art.

### SUMMARY OF THE INVENTION

The current invention is directed to methods, devices and systems for assessing appendicitis in a subject. Therefore, in one aspect, provided herein is a method for assessing appendicitis in a subject, which method comprises: a) determining values of a plurality of biomarkers in a sample from a subject; b) combining said values of said biomarkers using a mathematical algorithm to produce a numerical test score; and c) comparing said test score to a reference value to assess appendicitis in said subject.

In another aspect, provided herein is a device or system for assessing appendicitis in a subject, which device or system comprises: a) means for determining values of a plurality of biomarkers in a sample from a subject; and b) a computer readable medium containing executable instructions that when executed combine said values of said biomarkers using a mathematical algorithm to produce a numerical test score.

In still another aspect, provided herein is a method for assessing appendicitis in a subject, which method comprises: a) separating a subject into a group of having high risk for appendicitis or a group of having low risk for appendicitis; b) determining values of a plurality of biomarkers in a sample from said subject; and c) comparing said values of said biomarker to a reference value of the corresponding group to assess appendicitis in said subject.

In yet another aspect, provided herein is a kit or device for assessing appendicitis in a subject, which kit or device comprises: a) means for assessing appendicitis risk in a subject; and b) means for determining value of a biomarker in a sample from said subject.

In some embodiments, the present invention uses mathematical algorithms to diagnose whether a patient has appendicitis. The choice of biomarker measurements are used as input to these algorithms, and the means of determining the parameters are used in these algorithms.

In some embodiments, the biomarkers used to build classifiers in the present invention comprise absolute neutrophil count (ANC), percent neutrophil, white blood cell count (WBC), C-reactive protein (CRP), MRP 8/14, SAA1 and/or SAA2, hyaluronan, and MMP-9. All possible combinations of five or fewer of these biomarkers are examined during the selection process. The parameters for each classifier are determined by means dependent on the classifier model being used. The values of the parameters found by these means depend on the sample set used for training.

In some embodiments, the initial selection criterion is for high values of area under the ROC curve (AUROC). Classifiers that perform well for initial selection are verified using various bootstrap methods, and validated with a sample set that have no overlap with the training set. During verification, classifiers are considered to have performed well when the confidence intervals for sensitivity and specificity are small. During validation, classifiers are considered to have performed well when the sensitivity and specificity for the validation sample set are near to, or better, than the sensitivity and specificity found for the training set.

In some embodiments, the classifier models examined are Naïve Bayesian Classifiers (NBC), Fisher Linear Discriminants (FLD), and/or Logistic Regression (LR) models. All showed similar performance during selection, but the FLD and LR models behaved better during verification.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates the relationship among CBC, WBC and ANC.

FIG. 2 shows the AUC and discriminability of exemplary biomarkers. SAA refers to SAA 1 in FIG. 2.

FIG. 3 shows the assay sensitivity, specificity and negative predicable value of exemplary combinations of two biomarkers. SAA refers to SAA 1 in FIG. 3.

FIG. 4 shows the assay sensitivity, specificity and negative predicable value of exemplary combinations of three biomarkers. SAA refers to SAA 1 in FIG. 4.

FIG. 5 shows properties of an exemplary four marker combination assay.

FIG. 6 shows properties of exemplary two, three and four marker combination assays.

FIG. 7 shows additional properties of exemplary two, three and four marker combination assays. CP-11 and AB08 refer to two separate clinical studies. This data reflects performance of the listed biomarker combinations where CP-11 data was used to train or construct an algorithm and the AB08 performance was determined by applying the CP-11 derived algorithm to the AB08 data. This is a very stringent way to establish performance of a multi-marker panel.

FIG. 8 shows age distribution of patients in the study described in Example 1.

FIG. 9 shows patient disposition in the study described in Example 1.

FIG. 10 shows biomarker results by duration of symptoms in the study described in Example 1.

FIG. 11 shows ROC curve in the study described in Example 1.

FIG. 12 shows diagnosis and disposition by biomarker results in the study described in Example 1.

FIG. 13 shows AppyScore distributions of AA(+) and AA(−) subjects in the study described in Example 2.

FIG. 14 shows performance measures for the cohort in the study described in Example 2. Left panel: ROC curves for AppyScore. Right panel: Performance summaries for AppyScore with a cut-off of 4 based on the initial pre-clinical study cohort samples.

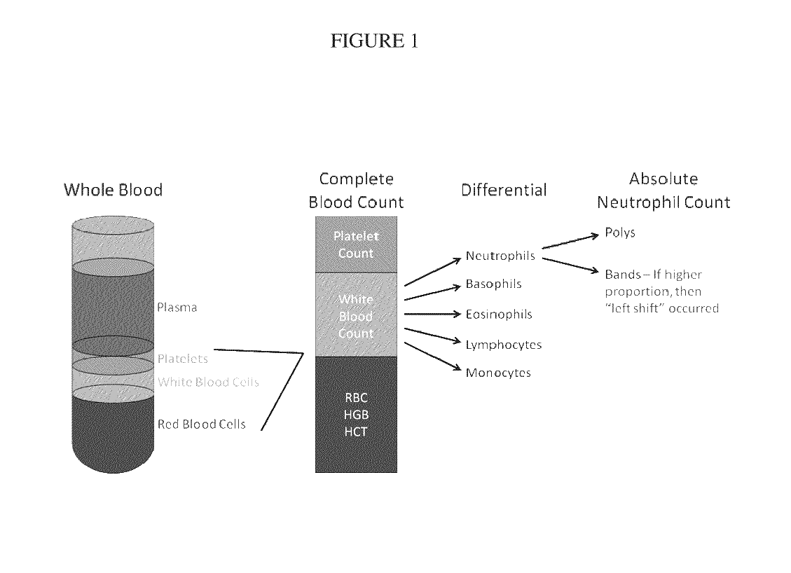

### DETAILED DESCRIPTION OF THE INVENTION

### A. Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a”, “an”, and “the” include plural references unless indicated otherwise. For example, “a” dimer includes one or more dimers.

The terms “polypeptide”, “oligopeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab', F(ab′)2, Fv), single chain (ScFv), mutants thereof, naturally occurring variants, fusion proteins comprising an antibody portion with an antigen recognition site of the required specificity, humanized antibodies, chimeric antibodies, single chain antibodies, and any other modified configuration of the immunoglobulin molecule that comprises an antigen recognition site of the required specificity. An “antibody” may be naturally occurring or man-made such as monoclonal antibodies produced by conventional hybridoma technology, various display methods, e.g., phage display, and/or a functional fragment thereof.

As used herein, the term “specific binding” refers to the specificity of a binding reagent, e.g., an antibody, such that it preferentially binds to a target antigen, such as myeloid related protein 8/14 (MRP 8/14), C-reactive protein (CRP), hyaluronan (HA), matrix metalloproteinase-9 (MMP-9), serum amyloid A1 (SAA 1), serum amyloid A2 (SAA 2), α1-antitrypsin (A1AT), epidermal growth factor (EGF), endothelial-leukocyte adhesion molecule 1 (ELAM-1 or E-Selectin), granulocyte colony-stimulating factor (G-CSF or GCSF), glutathione s-transferase omega-1 (GSTO1), interleukin-6 (IL-6), interleukin-8 (IL-8), junction plakoglobin (JUP), Layilin, lectin, galactose binding, soluble 3 (Lgals3), malate dehydrogenase (MDH or MADH), matrix metalloproteinase-1 (MMP-1), neural cell adhesion molecule 1 (NCAM 1), nuclear factor NF-kappa-B p105 subunit (NFKB-1), plasminogen activator inhibitor-1 (PAI-1), Parkinson disease (autosomal recessive, early onset) 7 (Park-7), procalcitonin (PCT), metallopeptidase inhibitor 1 (TIMP-1), urokinase-plasminogen activator (UPA), and vascular endothelial growth factor D (VEGF-D). Recognition by a binding reagent or an antibody of a particular target in the presence of other potential interfering substances is one characteristic of such binding. Preferably, binding reagents, antibodies or antibody fragments that are specific for or bind specifically to a target antigen bind to the target antigen with higher affinity than binding to other non-target substances. Also preferably, binding reagents, antibodies or antibody fragments that are specific for or bind specifically to a target antigen avoid binding to a significant percentage of non-target substances, e.g., non-target substances present in a testing sample. In some embodiments, binding reagents, antibodies or antibody fragments of the present disclosure avoid binding greater than about 90% of non-target substances, although higher percentages are clearly contemplated and preferred. For example, binding reagents, antibodies or antibody fragments of the present disclosure avoid binding about 91%, about 92%, about 93%, about 94%, about 95%, about 96%, about 97%, about 98%, about 99%, and about 99% or more of non-target substances. In other embodiments, binding reagents, antibodies or antibody fragments of the present disclosure avoid binding greater than about 10%, 20%, 30%, 40%, 50%, 60%, or 70%, or greater than about 75%, or greater than about 80%, or greater than about 85% of non-target substances.

As used herein, “biological sample” refers to any sample obtained from a living or viral source or other source of macromolecules and biomolecules, and includes any cell type or tissue of a subject from which nucleic acid or protein or other macromolecule can be obtained. The biological sample can be a sample obtained directly from a biological source or a sample that is processed. For example, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals and plants and processed samples derived therefrom.

It is understood that aspects and embodiments of the invention described herein include “consisting” and/or “consisting essentially of” aspects and embodiments.

Other objects, advantages and features of the present invention will become apparent from the following specification taken in conjunction with the accompanying drawings.

### B. Methods, Devices and Systems for Assessing Appendicitis in a Subject

The current invention is directed to methods, devices and systems for assessing appendicitis in a subject. Therefore, in one aspect, provided herein is a method for assessing appendicitis in a subject, which method comprises: a) determining values of a plurality of biomarkers in a sample from a subject; b) combining said values of said biomarkers using a mathematical algorithm to produce a numerical test score; and c) comparing said test score to a reference value to assess appendicitis in said subject.

The values of the biomarkers can be determined by any suitable ways. For example, the values of the biomarkers can be determined by determining amounts, concentrations and/or activities of the biomarkers.

Any suitable biomarkers can be used in the present methods. In some embodiments, the biomarkers used in the present methods are myeloid related protein 8/14 (MRP 8/14), C-reactive protein (CRP), hyaluronan (HA), matrix metalloproteinase-9 (MMP-9), serum amyloid A1 (SAA 1), serum amyloid A2 (SAA 2), a combination of SAA 1 and SAA 2, α1-antitrypsin (A1AT), epidermal growth factor (EGF), endothelial-leukocyte adhesion molecule 1 (ELAM-1 or E-Selectin), granulocyte colony-stimulating factor (G-CSF or GCSF), glutathione s-transferase omega-1 (GSTO1), interleukin-6 (IL-6), interleukin-8 (IL-8), junction plakoglobin (JUP), Layilin, lectin, galactose binding, soluble 3 (Lgals3), malate dehydrogenase (MDH or MADH), matrix metalloproteinase-1 (MMP-1), neural cell adhesion molecule 1 (NCAM 1), nuclear factor NF-kappa-B p105 subunit (NFKB-1), plasminogen activator inhibitor-1 (PAI-1), Parkinson disease (autosomal recessive, early onset) 7 (Park-7), procalcitonin (PCT), metallopeptidase inhibitor 1 (TIMP-1), urokinase-plasminogen activator (UPA), vascular endothelial growth factor D (VEGF-D), white blood cell count (WBC), absolute neutrophil count (ANC) and/or percent neutrophil in WBC (% NEU). In other embodiments, the combinations of the biomarkers used in the present methods do not include the combination of CRP and WBC, CRP and WBC, CRP and SAA, SAA and WBC, or CRP, SAA and WBC.

Any suitable number of biomarkers can be used in the present methods. For example, the values of at least 2, 3, 4, 5, 6, 7, 8, 9 or 10 biomarkers can be determined and combined into a test score.

In some preferred embodiments, any combinations of the following biomarkers can be determined and combined into a test score: myeloid related protein 8/14 (MRP 8/14), C-reactive protein (CRP), hyaluronan (HA), matrix metalloproteinase-9 (MMP-9), serum amyloid A1 (SAA 1), serum amyloid A2 (SAA 2), white blood cell count (WBC), absolute neutrophil count (ANC) and/or percent neutrophil in WBC (% NEU). For example, the values of at least 2, 3, 4, or 5 of the above biomarkers can be determined and combined into a test score.

In some preferred embodiments, the following combinations of the biomarkers can be determined and combined into a test score: ANC and CRP; ANC and HA; ANC and MMP-9; ANC and MRP 8/14; ANC and % NEU; ANC and SAA; ANC and WBC; CRP and HA; CRP and MMP-9; CRP and MRP 8/14; CRP and % NEU; CRP and SAA; CRP and WBC; HA and MMP-9; HA and MRP 8/14; HA and % NEU; HA and SAA; HA and WBC; MMP-9 and MRP 8/14; MMP-9 and % NEU; MMP-9 and SAA; MMP-9 and WBC; MRP 8/14 and % NEU; MRP 8/14 and SAA, MRP 8/14 and WBC, % NEU and SAA; % NEU and WBC; SAA and WBC; ANC, CRP and HA; ANC, CRP and MMP-9; ANC, CRP and MRP 8/14; ANC, CRP and % NEU; ANC, CRP and SAA; ANC, CRP and WBC; ANC, HA and MMP-9; ANC, HA and MRP 8/14; ANC, HA and % NEU; ANC, HA and SAA; ANC, HA and WBC; ANC, MMP-9 and MRP 8/14; ANC, MMP-9 and % NEU; ANC, MMP-9 and SAA; ANC, MMP-9 and WBC; ANC, MRP 8/14 and % NEU; ANC, MRP 8/14 and SAA; ANC, MRP 8/14 and WBC; ANC, % NEU and SAA; ANC, % NEU and WBC; ANC, SAA and WBC; CRP, HA and MMP-9; CRP, HA and MRP 8/14; CRP, HA and % NEU; CRP, HA and SAA; CRP, HA and WBC; CRP, MMP-9 and MRP 8/14, CRP, MMP-9 and % NEU; CRP, MMP-9 and SAA; CRP, MMP-9 and WBC; CRP, MRP 8/14 and % NEU; CRP, MRP 8/14 and SAA; CRP, MRP 8/14 and WBC; CRP, % NEU and SAA; CRP, % NEU and WBC; CRP, SAA and WBC; HA, MMP-9 and MRP 8/14; HA, MMP-9 and % NEU; HA, MMP-9 and SAA; HA, MMP-9 and WBC; HA, MRP 8/14 and % NEU; HA, MRP 8/14 and SAA; HA, MRP 8/14 and WBC; HA, % NEU and SAA; HA, % NEU and WBC; HA, SAA and WBC; MMP-9, MRP 8/14 and % NEU; MMP-9, MRP 8/14 and SAA; MMP-9, MRP 8/14 and WBC; MMP-9, % NEU and SAA; MMP-9, % NEU and WBC; MMP-9, SAA and WBC; MRP 8/14, % NEU and SAA; MRP 8/14, % NEU and WBC; MRP 8/14, SAA and WBC; % NEU, SAA and WBC. SAA can specify SAA 1, SAA 2 or a combination of SAA 1 and SAA 2.

In some embodiments, the myeloid related protein 8/14 (MRP 8/14) biomarker is a heterodimer protein complex composed of individual myeloid-related proteins, MRP8 and MRP14, otherwise individually known as S100A8 or calgranulin A and S100A9 or calgranulin B, respectively. These intracellular calcium binding proteins are key to the transduction of calcium signaling during the inflammation process, Passey, R J, K Xu, DA Hume, and CL Geczy. “S100A8: emerging functions and regulation.” J Leukoc Biol 66, no. 4 (October 1999): 549-56.

Together, MRP 8/14 is sometimes referred to as calprotectin, and is expressed in neutrophils, monocytes, some epithelial cells, and keratinocytes of inflamed tissues. Most of MRP 8/14's proinflammatory functions require extracellular release, but the exact secretory mechanism is not fully understood. What is understood is that this mechanism is tightly controlled, requires activation of two independent signally pathways, and are highly over-secreted during the inflammatory process, Rammes, A, J Roth, M Goebeler, M Klempt, M Hartmenn, and C Sorg. “Myeloid-related protein (MRP) 8 and MRP14, calcium-binding proteins of the S100 family, are secreted by activated monocytes via a novel, tubulin-dependent pathway.” J Biol Chem 272, no. 14 (April 1997): 9496-502. Circulating levels of MRP8/14 have been shown to be increased during acute appendicitis and could potentially differentiate acute appendicitis from non-inflammatory-related abdominal pain.

MRP 8/14 has been studied for appendicitis. For example, Beater and Colgin. S100A8/A9: A Potential New Diagnostic Aid for Appendicitis. Acad Emerg Med. 2010; 17:333-336, evaluates the effectiveness of calgranulin in aiding in the diagnosis of appendicitis. In another example, Thujils et al. A pilot study on potential new plasma markers for diagnosis of acute appendicitis. Am. J. Emerg. Med. 2011 March; 29(3):256-60 evaluate the effectiveness of calgranulin (MRP 8/14), lactoferrin, c-reactive protein, and white blood count in aiding in the diagnosis of appendicitis.

In some embodiments, C-Reactive Protein (CRP) is a pentameric protein consisting of five 25 kD monomers that is produced in hepatocytes. The levels of CRP in blood increase in response to inflammation, in some cases up to 10,000 fold, Pepys, Mark B, and Gideon M Hirschfield. “C-reactive protein: a critical update.” J. Clin Invest 111, no. 12 (2003): 1805-1812. This increase develops in a wide range of acute and chronic inflammatory conditions. These conditions promote the release of interleukin-6 that triggers the transcriptionally controlled synthesis of CRP by the liver. During the acute phase response, levels of CRP rapidly increase within 2 hours of acute insult, reaching a peak at 48 hours. With resolution of the acute phase response, CRP declines with a relatively short half-life of 19 hours, Vigushin, DM, MB Pepys, and PN Hawkins. “Metabolic and Scintigraphic Studies of Radioiodinated Human C-reactive Trotein in Health and Disease.” J. Clin. Invest. 91 (1993): 1351-1357. Measuring CRP level is a screen for infectious and inflammatory diseases. Numerous clinical studies have been conducted concluding that CRP is a good candidate as a biomarker for appendicitis. Notably, CRP is only meaningful after 24 hours of symptoms being present.¹⁸

Some studies using CRP in combination with another factor (white blood count, absolute neutrophil count, another protein biomarker, clinical prediction rule) specifically for appendicitis have been reported. For example, some studies using CRP in combination with ANC or WBC. See e.g., Andersson. Meta-analysis of the clinical and laboratory diagnosis of appendicitis. Brit J of Surg. 2004; 91:28-37; Beltran et al. Predictive value of white blood cell count and C-reactive protein in children with appendicitis. J Ped Surg. 2007; 42: 1208-1214; Birchley. Patients with clinical acute appendicitis should have pre-operative full blood count and C-reactive protein assays. Ann R Coll Surg Engl. 2006; 88: 27-32; Gronroos et al. Phospholiapase A₂, C-Reactive Protein, and White Blood Cell Count in the Diagnosis of Acute Appendicitis. Clin Chem. 1994; 40(9): 1757-1760; Kim et al. C-reactive protein estimation does not improve accuracy in the diagnosis of acute appendicitis in pediatric patients. Int J. Surg. 2009; 7: 74-77; Kwan and Nager. Diagnosing pediatric appendicitis: usefulness of laboratory markers. Amer J of Emerg Med. 2010; 28: 1009-1015; Mohammed et al. The diagnostic value of C-reactive protein, white blood cell count and neutrophil percentage in childhood appendicitis. Saudi Med J. 2004; 25(9): 1212-1215; Ortega-Deballon et al. Usefulness of Laboratory Data in the Management of Right Iliac Fossa Pain in Adults. Dis Colon Rectum. 2008; 51(7): 1093-99; Rodriguez-Sanjuan et al. C-Reactive Protein and Leukocyte Count in the Diagnosis of Acute Appendicitis in Children. Dis Colon Rectum 1999; 42(10): 1325-9; Sengupta et al. White cell count and c-reactive protein measurement in patients with possible appendicitis. Ann R Coll Surg Engl. 2009; 91: 113-5; Siddique et al. Diagnostic accuracy of white cell count and C-reactive protein for assessing the severity of paediatric appendicitis. J R Soc Med Sh Rep. 2011; 2(57): 59; Van Diejen-Visser et al. The Value of Laboratory Tests in patients Suspected of Acute Appendicitis. Eur J Clin Chem Clin Biochem. 1991; 29: 749-52; Vaughan-Shaw et al. Normal inflammatory markers in appendicitis: evidence from two independent cohort studies. J R Soc Med Sh Rep. 2011; 2: 43; and Yildrim et al. The Role of Serum Inflammatory Markers in Acute Appendicitis and Their Success in Preventing Negative Laparotomy. J Invest Surg. 2006; 19: 345-52.

Other studies using CRP in combination with other protein biomarkers. See e.g., Yildrim et al. The Role of Serum Inflammatory Markers in Acute Appendicitis and Their Success in Preventing Negative Laparotomy. J Invest Surg. 2006; 19: 345-52. Other studies using CRP in combination with clinical prediction rule (Modified Alvarado Score). See e.g., Shafi et al. Evaluation of the modified Alvarado score incorporating the C-reactive protein in the patients with suspected acute appendicitis. Ann Nigerian Med. 2011; 5(1): 6-11.

In some embodiments, hyaluronan (also called hyaluronic acid or hyaluronate) is an anionic, nonsulfated glycosaminoglycan or linear (unbranched) polysaccharide that forms in the plasma membrane and can often be very large. Hyaluronan (HA) is distributed widely throughout connective, epithelial, and neural tissues and is one of the chief components of the extracellular matrix. HA can be broken down into smaller fragments during inflammatory responses and can further promote immune responses, Scheibner K A, Lutz M A, Boodoo S, et al. “Hyaluronan Fragments Act as an Endogenous Danger Signal by Engaging TLR2.” J of Immunology, 2006: 177:1272-1281. HA has previously been studied as a biomarker for several disease indications including osteoarthritis, liver disease, pulmonary fibrosis and rheumatoid arthritis.

In some embodiments, MMPs are matrix metalloproteinase (MMPs), also known as matrixins, act as proteinases and play a central role in many biological processes. MMPs are responsible for hydrolyzing components of the extra cellular matrix and more specifically are involved in embryonic development, reproduction, and tissue remodeling, as well as in disease processes, such as arthritis and metastasis. MMP activity has been linked to tumor growth, invasion, and angiogenesis inflammation and may actually work in a non-proteolytic manner, Jun, Sun. “Matrix Metalloproteinases and Tissue Inhibitor of Metalloproteinases are Essential for the Inflammatory Response in Cancer Cells.” Journal of Signal Transduction 2010:9881532 (2010): 1-7. The upregulation of MMPs is part of the pathway leading to tissue damage and perforation. Solberg et al. proposes that MMPs and tissue inhibitor of metalloproteinases (TIMPs) are involved in inflammatory conditions in the intestines, which may in turn suggest that MMPs would be useful as biomarkers for inflammatory disease, Solberg, A, L Holmdahl, P Falk, I Palmgren, and M L Ivarsson. “A local imbalance between MMP and TIMP may have an implication on the severity and course of appendicitis.” Int J Colorectal Dis 23(6) (2008): 611-8.

Because the destruction of the extracellular matrix precedes perforation, it has been suggested that MMP-9 can help predict appendix perforation. Several studies have directly examined MMP-9 in appendicitis. For example, Dalal et al. Serum and Peritoneal Inflammatory Mediators in Children With Suspected Acute Appendicitis. Arch Surg. 2005; 140: 169-173 evaluates the value of various biomarkers, including MMP-9, in diagnosing appendicitis; Solberg et al. A local imbalance between MMP and TIMP may have an implication on the severity and course of appendicitis. Int J Colorectal Dis. 2008; 23: 611-618 evaluates the ability of MMP-9, and other markers, to predict the severity of appendicitis; Ruber et al. Systemic Th17-like cytokine pattern in gangrenous appendicitis but not in phlegmonous appendicitis. J. Surg. 2010; 147: 366-72 investigates differences in gangrenous and phlegmonous appendicitis, including MMP-9 concentrations; Solberg et al. Tissue Proteolysis in Appendicitis with Perforation. J of Surg Research. 2010 investigates proteolysis in appendices that have perforated, including the role of MMP-9.

In some embodiments, SAA Serum amyloid A (SAA) makes up a family of differentially expressed apolipoproteins in which there is a high degree of homology between species. These proteins are categorized as acute-phase or constitutive SAAs and are primarily synthesized in the liver. Acute-phase can increase 1.000-fold during inflammation, whereas constitutive SAAs are only induced minimally during inflammation. The cytokines, IL-1 and IL-6, are involved in pathways that cue for the induction of SAA upon pro-inflammatory stimuli. SAA roles may include involvement in lipid metabolism and transport, induction of extracellular-matrix-degrading enzymes, and recruitment of inflammatory cells to the inflammation site, Uhlar, C M, and A S Whitehead. “Serum amyloid A, the major vertebrate acute-phase reactant.” Eur J Biochem 265, no. 2 (October 1999): 501-23. SAA is as sensitive as CRP for acute-inflammation marker, but not used as readily as a target marker for inflammation as CRP, Malle, E, Steinmetz, A and J G Raynes. “Serum amyloid A (SAA): an accute phase protein and apolipoprotein.” Atherosclerosis 102, no. 2 (September 1993): 131-46.

By being a part of the acute inflammatory response, increased SAA levels will appear quickly (within minutes to hours) and will only be present as long as there is inflammation. This early role in inflammation and specificity to the acute-phase make SAA a potentially useful biomarker for acute appendicitis. For example, Lycopoulou et al. Serum amyloid A protein levels as a possible aid in the diagnosis of acute appendicitis in children. Clin Chem Lab Med. 43(1):49-53. 2005 compared the sensitivity and specificity of SAA, CRP, and WBC in pediatrics with confirmed appendicitis.

In some embodiments, A1AT is a 52-kDa serine protease inhibitor (serpin) involved in the acute inflammatory response. Upon exposure to inflammatory stimuli, the concentration of A1AT rises rapidly. A1AT prevents tissue damage by inhibiting neutrophil elastase, chemotaxis, and adhesion. Additionally, A1AT inhibits apoptosis by direct inhibition of the intracellular cysteine protease caspase-3, Petrache, I, et al. “alpha-1 antitrypsin inhibits caspase-3 activity, preventing lung endothelial cell apoptosis.” Am J Pathol 169, no. 4 (October 2006): 1155-66.

In some embodiments, epidermal growth factor receptor (EGFR) is a cell-surface receptor which binds ligands that are members of the epidermal growth factor family. (Herbst 2004) Binding of the inactive monomeric receptor to its ligand induces dimerization and activates the receptor, Yarden, Y, and J. Schlessinger. “Epidermal growth factor induces rapid, reversible aggregation of the purified epidermal growth factor receptor.” Biochemistry 26, no. 5 (March 1987): 1443-51. Dimerization induces autophosphorylation of specific C-terminal EGFR tyrosine residues and initiates signal transduction of the MAPK, JNK, and Akt pathways, leading to DNA synthesis and cell proliferation, Oda, K, Y Matsuoka, A Funahashi, and H Kitano. “A comprehensive pathway map of epidermal growth factor receptor signaling.” Mol Syst Biol 1 (2005): 2005.0010. Mutations affecting EGFR expression or activity could result in cancer.

In some embodiments, E-selectin, also known as endothelial leukocyte adhesion molecule-1 (ELAM-1) or CD62E, is exclusively expressed by cytokine-activated endothelial cells. E-selectin is a 110 kD protein activated by cyokines (TNF, IL-1) during inflammatory reactions. It recognizes complex sialylated carbohydrate groups found on surface proteins of monocytes, graunulocytes, and previously activated T cells. Its primary function is to home effector and memory T cells to some peripheral sites of inflammation—particularly the skin.

In some embodiments, Granulocyte Colony-Stimulating Factor (GCSF) is a 19 kD hematopoietic cytokine produced by macrophages, endothelial cells, and fibroblasts. Its principal targets are bone marrow progenitors. GCSF promotes maturation of bone marrow cells into granulocytes and monocytes in response to infection and inflammatory activity, Allister, L, R Bachur, J Glickman, and B Horwitz. “Serum markers in acute appendicitis.” J Surg Res 168, no. 1 (June 2011): 70-5.

In some embodiments, Glutathione S-Transferase Omega 1 (GSTO1) is a 27 kDa enzyme which is part of the GST superfamily. In humans, GSTO1 is expressed in most tissues and exhibits both glutathione-dependent thiol transferase and dehydroascorbate reductase activities. Unlike other mammalian GSTs, GSTO 1-1 appears to have an active site cysteine that can form a disulfide bond with glutathione. (Board, et al. 2000) Board, P G, et al. “Identification, characterization, and crystal structure of the Omega class glutathione transferases.” 275, no. 32 (August 2000): 24798-24806. Human GSTOs catalyze thioltransferase reactions and play a role in protein glutathionylation reactions, Whitbread, A K, A Masoumi, N Tetlow, E Schmuch, M Coggan, and Board P G. “Characterization of the omega class of glutathione transferases.” Methods Enzymol 401 (2005): 78-99. GSTOs display dehydroascorbate reductase activity, which involves them in cellular antioxidant defense mechanisms, Ishikawa, T, A F Casini, and M Nishikimi “Molecular cloning and functional expression of rat liver glutathione-dependent dehydroascorbate reductase.” J Biol Chem 273, no. 44 (October 1998): 28708-12 and Xu, D P, M P Washburn, G P Sun, and W W Wells. “Purification and characterization of a glutathione dependent dehydroascorbate reductase from human erythrocytes.” Biochem Biophys Res Commun 222, no. 1 (April 1996): 117-21.

In some embodiments, Interleukin-6 (IL-6) is a pleiotropic cytokine involved in provoking a broad range of cellular and physiological responses. IL-6 is a critical mediator of the acute phase response. IL-6 is released from macrophages and T cells to stimulate immune response to infection, burns, trauma, neoplasia, and other tissue damage leading to inflammation. IL-6 can act in response as both a pro-inflammatory and anti-inflammatory cytokine. IL-6 is in part responsible for stimulating and regulating acute phase protein synthesis, as well stimulating increased production of neutrophils in the bone marrow. Additionally, IL-6 stimulates B cell proliferation and differentiation, but inhibits regulatory T cells. IL-6 levels have previously been reported to correlate with appendiceal inflammation, Sack, U, B Biereder, K Bauer, T Keller, and RB Trobs. “Diagnostic value of blood inflammatory markers for detection of acute appendicitis in children.” BMC Surg 6, no. 15 (November 2006).

In some embodiments, Interleukin-8 (IL-8) is proinflammatory CXC chemokine produced by several types including macrophages, neutrophils, epithelial cells, endothelial cells. IL-8 is well known for its effects on neutrophils, particularly its ability to function as a chemoattractant to sites of inflammation. In addition to IL-8 effects on neutrophils, it promotes angiogenesis, inhibits endothelial cell apoptosis, and promotes the proliferation of melanoma cells in an autocrine fashion. (Payne and Cornelius 2002) Payne, A S, and L A Cornelius. “The role of chemokines in melanoma tumor growth and metastasis.” J Invest Dermatol 118, no. 6 (June 2002): 915-22. IL-8 levels have previously been shown to be elevated in urine, peritoneal fluid and serum from patients undergoing appendicitis, Allister, L, R Bachur, J Glickman, and B Horwitz. “Serum markers in acute appendicitis.” J Surg Res 168, no. 1 (June 2011): 70-5, Taha, A S, Grant, V, and R W Kelly. “Urinalysis for interleukin-8 in the non-invasive diagnosis of acute and chronic inflammatory diseases.” Postgrad Med J 79, no. 929 (March 2003): 159-63, and Zeillemaker, A M, A A Hoynck van Papendrecht, M H Hart, D Roos, H A Verbrugh, and P Leguit. “Peritoneal interleukin-8 in acute appendicitis.” J Surg Res 62, no. 2 (May 1996): 273-7.

In some embodiments, JUP, also known as junction plakoglobin or gamma-catenin, is a member of the Armadillo family of signaling molecules. JUP localizes to both desmosomes and adherens junctions, which are sites of cell adhesion. JUP interacts with the cytoplasmic domains of cadherins and desmosomal cadherins.

In some embodiments, layilin is an integral membrane hyaluronan receptor located in the membrane ruffles that interacts with merlin and radixin, Hynes, R O et al., “Layilin, a cell surface hyaluronan receptor, interacts with merlin and radixin.” Ex Cell Res. 308, no. 1 (2005): 177-87. Layilin is a talin-binding transmembrane protein that is homologous with C-type lectins, Borowsky, M L and Hynes, R O. “Layilin, a novel talin-binding transmembrane protein homologous with C-type lectins, is localized in membrane ruffles.” J Cell Biol 143, no. 2 (1998): 429-442.

In some embodiments, galectin (or LGAL) proteins function as beta-galactoside-binding proteins. Galectin proteins are thought to play a role in numerous cellular functions, including cell-cell and cell-matrix interactions, cell adhesion, apoptosis, innate immunity, T-cell regulation, and possibly cell proliferation, Rabinovich G A, Baum, L G, Tinari N, Paganelli R, Natoli C, Liu F T, Iacobelli, S. “Galectins and their ligands: amplifiers, silencers or tuners of the inflammatory response?” Trends Immunol. 23, no. 6 (2002): 313-30, Grusen, D and G Ko. “Galectins testing: new promises for the diagnosis and risk stratification of chronic diseases?” Clin Biochem 45, no. 10-11 (2012): 719-26, and Leffler H, Carlsson S, Hedlund M, Qian Y, Poirier F. “Introductions to galectins.” Glycoconj J. 19, no. 7-9 (2004): 433-30. Additionally, the galectin family of proteins are characterized by a proline-rich tandem repeat on the N-terminus, Barboni E A, Bawumia S, Henrick K, Hughes, RC. “Molecular modeling and mutagenesis studies of the N-terminal domains of galectin-3: evidence for participation with the C-terminal carbohydrate recognition domain in oligosaccharide binding.” 10, no. 11 (2000): 1201-8.

In some embodiments, malate dehydrogenase (MDH) is a protein complex that functions as an enzyme to catalyze the conversion of malate to oxaloacetate in the citric acid cycle. MDH has also been implicated in gluconeogenesis. Additionally, MDH mRNA has been shown to be upregulated 30-fold in response to LPS (endotoxin), suggesting its possible role in bacterial infection, Suzuki T, Hashimoto S, Toyoda N, Nagai S, Yamazaki N, Dong H Y, Sakai J, Yamashita T, Nukiwa T, Matsushima K. “Comprehensive gene expression profile of LPS-stimulated human monocytes by SAGE.” Blood 96, no. 7 (2000): 2584-91.

In some embodiments, matrix metalloproteinase-1 (MMP-1) is an enzyme that functions in the breakdown of the extracellular matrix of cells. MMP-1 activity has been noted in physiological processes such as tissue growth, reproduction, embryonic development, as well as several disease processes, Clark I M, Swingler T E, Sampieri C L, Edwards D R. “The regulation of matrix metalloproteinases and their inhibitors.” Int J Biochem 40, no. 6-7 (2008): 40. Additionally, MMP-1 proteins are activated following cleavage by extracellular proteases.

In some embodiments, neural cell adhesion molecule (NCAM) is expressed on the surface of skeletal muscle, natural killer cells, neurons, and glia, functioning as a homophilic binding glycoprotein, Cunningham B A, Hemperly J J, Murray B A, Prediger E A, Brackenbury R, Edelman G M. “Neural cell adhesion molecule: structure, immunoglobulin-like domains, cell surface modulation, and alternative RNA splicing.” Science 236, no. 4803 (1987): 799-806, Seidenfaden R, Krauter A, Schertzinger F, Gerardy-Schahn R, Hildebrandt H. “Polysialic acid directs tumor cell growth by controlling heterophilic neural cell adhesion molecule interactions.” Mol Cell Bio 23, no. 16 (2003): 5908-18 and Robertson M J, Ritz J. “Biology and clinical relevance of human natural killer cells.” Blood 76, no. 12 (1990): 2421-38. NCAM is thought to be associated with learning, memory, cell-cell adhesion, synaptic plasticity, and neurite outgrowth (by activating the fibroblast growth factor receptor and the p59Fyn signaling pathway), Schmid R S, Graff R D, Schaller M D, Chen S, Schachner M, Hemperly J J, Maness P F. “NCAM stimulates the Ras-MAPK pathway and CREB phosphorylation in neuronal cells.” J Neurobiol 38, no. 4 (1999): 542-58.

In some embodiments, NFκB regulates the transcription of DNA, functioning as a protein heterodimer. NFκB, when incorrectly regulated, has been linked to autoimmune diseases, septic shock, viral infections, and cancer, Brown K D, Claudio E, Siebenlist U. “The roles of the classical and alternative nuclear factor-kappaB pathways: potential implications for autoimmunity and rheumatoid arthritis.” Arthritis Res Ther 10, no. 4 (2008): 212. Escarcega R O, Fuentes-Alexandro S, García-Carrasco M, Gatica A, Zamora A. “The transcription factor nuclear factor-kappa B and cancer.” Clin Oncol (R Coll Radiol) 19, no. 2 (2007): 154-61 and Jimi E and Ghosh S. “Role of nuclear factor-kappaB in the immune system and bone.” Immunol Rev 208 (2005): 80-7. NFκB has also been shown to be associated with numerous cellular responses, such as those induced by ultraviolet irradiation, free radicals, bacterial/viral antigens, and even stress. Additionally, NFκB functions in the processes of plasticity and memory of synapses.

In some embodiments, PAI-1 functions as a serine protease inhibitor, acting as the main inhibitor of the fibrinolysis pathway (the breakdown of blood clots), Iwaki T, Urano T, Umemura K. “PAI-1, progress in understanding the clinical problem and its aetiology.” Br J Haematol 157, no. 3 (2012): 291-8. PAI-1 is primarily produced in the endothelium, though secretion has also been observed in other tissues types, such as adipose tissue. PAI-1 has also been implicated in the inhibition of matrix metalloproteinases, which function to invade malignant cells across the basal lamina.

In some embodiments, PARK7 is a peptidase belonging to the C56 protein family. PARK7 is thought to function as a sensor for oxidative stress by acting as a redox-sensitive chaperone. PARK7 has been shown to be a positive regulator of androgen receptor-dependant transcription, Tillman J E, Yuan J, Gu G, Fazli L, Ghosh R, Flynt A S, Gleave M, Rennie P S, Kasper S. “DJ-1 binds androgen receptor directly and mediates its activity in hormonally treated prostated cancer cells.” Cancer Res 67, no. 10 (2007): 4630-7. Additionally, PARK7 may protect against the oxidative stress and cell death in neurons, Marcondes A M, Li X, Gooley T A, Milless B, Deeg H J. “Identification of DJ-1/PARK-7 as a determinant of stroma-dependent and TNF-alpha-induced apoptosis in MDS using mass spectrometry and phosphopeptide analysis.” Blood 115, no. 10 (2010): 1993-2002.

In some embodiments, PCT is a peptide precursor of the hormone calcitonin, the latter being involved with calcium homeostasis. It is composed of 116 amino acids and is produced by parafollicular cells (C cells) of the thyroid and by the neuroendocrine cells of the lung and the intestine. Procalcitonin can be used as a biomarker for sepsis. McGee K A, Baumann, N A. Procalcitonin: Clinical Utility in Diagnosing Sepsis. Clin. Lab. News 35(7). The level of procalcitonin in the blood stream of healthy individuals is below the limit of detection (10 pg/mL) of clinical assays. The level of procalcitonin raises in a response to a proinflammatory stimulus, especially of bacterial origin. It does not rise significantly with viral or non-infectious inflammations. With the derangements that a severe infection with an associated systemic response brings, the blood levels of procalcitonin may rise to 100 ng/ml.

In some embodiments, TIMP1, a tissue inhibitor of metalloproteinases, is a glycoprotein that is expressed from the several tissues of organisms. The glycoprotein is a natural inhibitor of the matrix metalloproteinases (MMPs), a group of peptidases involved in degradation of the extracellular matrix. In addition to its inhibitory role against most of the known MMPs, the encoded protein is able to promote cell proliferation in a wide range of cell types, and may also have an anti-apoptotic function. Transcription of this gene is highly inducible in response to many cytokines and hormones. TIMP1 has been associated with appendicitis, Solberg A, Holmdahl L, Falk P, Wolving M, Palgren I, Ivarsson M L. “Local and systemic expressions of MMP-9, TIMP-1 and PAI-1 in patients undergoing surgery for clinically suspected appendicitis.” 48, no. 2 (2012): 99-105.

In some embodiments, uPA, urokinase plasminogen activator and its associated receptor are the target of many cancer therapies. uPA is a serine protease associated with breast cancer, it is implicated in cancer invasion and metastasis, Duffy M J, Reilly D, O'Sullivan C, O'Higgins N, Fennelly J J, Andreasen P. “Urokinase-plasminogen activitor, a new and independent prognostic marker in breast cancer.” Cancer Res 50, no. 21 (1990): 6827-9.

In some embodiments, VEGF is a signal protein produced by cells that stimulates vasculogenesis and angiogenesis. It is part of the system that restores the oxygen supply to tissues when blood circulation is inadequate, Olsson A K, Dimberg A, Kreuger J, Claesson-Welsh L. “VEGF receptor signalling—in control of vascular function.” Nat Rev Mol Cell Bio 7, no. 5 (2006): 359-71. VEGF's normal function is to create new blood vessels during embryonic development, new blood vessels after injury, muscle following exercise, and new vessels (collateral circulation) to bypass blocked vessels. VEGF is believed to be involved with several types of cancers, Gatto B, Cavalli M. “From proteins to nucleic acid-based drugs: the role of biotech in anti-VEGF therapy.” Anticancer Agents Med Chem 6, no. 4 (2006): 287-301.

The most common laboratory test ordered for abdominal pain is the complete blood count (CBC). They are ordered 69% of the time resulting in 6.6 million a year for abdominal pain.¹ The test provides information on the number of red blood cells (RBC), hemoglobin (HGB), hematocrit (HCT), platelets, and importantly for appendicitis, white blood cells (WBC). A serum sample is transferred to the laboratory and placed into a cell counter. The cell counter utilizes radio frequency and electrical impedance in order to count and measure the various cells. Results can be reported within 10 minutes. However, typically, a WBC is only effective after 24 hours of symptoms being present.¹⁸

In some embodiments, white blood cells (WBCs) or leukocytes, are cells of the immune system that help the body in defending against infections. WBCs circulate in the blood stream with at typical concentration of 4,000-10,000 cells per microliter. Most WBCs are formed in the bone marrow from a multipotent cell called a hematopoietic stem cell. The five different types of WBCs are neutrophils, lymphocytes, monocytes, eosinophils and basophils. WBCs typically live for about 3 to 4 days in the average human body. An increase in the number of WBCs in circulation is often an indicator of disease. A WBC count contains information on the number of total WBCs, however, it is often important to determine what the “differential” values measure. The differential is a measurement of the five WBC types listed above. WBC count has been used over the years in attempts to aid in the diagnosis of appendicitis, however, the accuracy of this test is limited in children, Rothrock, SG, and J Pagane. “Acute appendicitis in children: emergency department diagnosis and management.” Ann Emerg Med 36, no. 1 (July 2000): 39-51.

In some embodiments, absolute neutrophil count (ANC) is a specific measure of the number of neutrophil granulocytes present in the blood. Neutrophils (also known as polymorphonuclear cells, PMN's, polys, granulocytes, segmented neutrophils or segs) are infection fighting leukocytes that are the most numerous leukocytes, generally 56% of the total amount of leukocytes.¹⁹ The ANC is included in the WBC differential. A deviation from a normal level is clinically significant. Neutropenia, a low ANC, is important as it signifies an increase risk of infection. Neutrophilia, a high ANC, is important as it is often indicative of the body mounting an immune response.

Importantly for appendicitis, the neutrophil category is further broken down into “polys” and “bands”. This is a measurement of the maturity of the neutrophils: “polys” are mature and “bands” are immature and important in fighting infections. In order to get the total number of neutrophils, the ANC is derived from the number of polys and bands. ANC is calculated by the following formula:

ANC=Total # WBC×(% polys+% bands) (In some cases, percentages are converted to decimals)

Furthermore, the physician is looking for a “left shift” of these neutrophils, meaning a higher proportion of bands. This observation communicates to the physician that the body is mounting an offensive against something (like appendicitis).

The present methods can be used for assessing appendicitis in a subject using any suitable sample obtained from the subject. For example, the sample can be a serum, a plasma and a blood sample. In another example, the sample can be a clinical sample. The present methods can be used for assessing appendicitis in any suitable subject. For example, the subject can be a human, and the biomarkers are corresponding human biomarkers. In some embodiments, the human subject is a male, female, an infant, a child, a teenager, a young adult, e.g., a young adult, less than 18, 21, 25 or 30 years old, a middle aged person or a senior.

The values of the biomarkers can be determined by any suitable reagents. For example, the values of the biomarkers can be determined using binding reagents that bind to, and preferably specifically bind to, the biomarkers. Exemplary binding reagents include antibodies, receptors, especially soluble receptors, and aptamers. The values of the biomarkers can be determined by any suitable methods. For example, the values of the biomarkers can be determined by an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, surface plasmon resonance (SPR), chemiluminescence assay, lateral flow immunoassay, u-capture assay, inhibition assay or avidity assay.

The values of the biomarkers can be combined using any suitable mathematical algorithm to produce a numerical test score. For example, the values of the biomarkers can be combined using Naïve Bayesian Classifiers (NBC), Fisher Linear Discriminants (FLD) and/or Logistic Regression (LR) to produce a numerical test score.

In some embodiments, Bayesian Classifiers make use of Bayes Theorem for predicting the likelihood that a sample x is a member of disease state sᵢ, given by p(sⱼ|x)=p(x|sⱼ) p(sⱼ)/p(x) where x=(x₁, x₂, x₃ . . . xᵢ . . . ), and xᵢ is the value of biomarker i. In words, the probability that a sample having biomarker values x comes from a patient in disease state sj, is equal to the probability that a patient in disease state sj has biomarker values of x, times the prevalence of disease state sj, divided by the probability that a patient regardless of disease state has biomarkers values of x. In practice, since p(x) is independent of disease state, this element may be ignored, as it provides no means of discrimination between disease states.

Since p(sⱼ), equivalent to the prevalence of each disease state within the population, is known in most cases, the problem then becomes one of calculating p(x|sⱼ). The Naïve Bayesian Classifier (NBC) assumes that the biomarkers are independent, so that the probability of the biomarker value vector x is simply the product of the individual biomarker probabilities:

P(x|sⱼ)=p(x₁|sⱼ)p(x₂|sⱼ) . . . p(xᵢ|sⱼ) . . . .

Although the assumption of independence is demonstrably invalid in most cases, it has been shown that the classifier models that make this assumption usually perform nearly as well as models that do not assume independence. The reduction in model complexity (and thus computational complexity) is therefore justified.

The most popular form for the NBC uses the normal distribution for the population density function:

p(xⱼ|sⱼ)=exp(−0.5*zⱼⱼ²)/(σᵢⱼ√(2π)), where z=(xᵢ−μᵢⱼ)/σᵢⱼ

μᵢⱼ is the average of biomarker i in disease state j, and σᵢⱼ is the standard deviation of biomarker I in disease state j. The function exp( ) refers to the constant e raised to the power of the argument.

The direct Bayesian discriminant would be equal to the likelihood p(sⱼ|x). However, the computational complexity can be reduced considerably by taking the natural log of the likelihood, then eliminating all terms that are constant between disease states:

gⱼ(x)=ln(p(sⱼ))−Σᵢ(zᵢⱼ²/2+ln(σᵢⱼ))

In some embodiments, Fisher Linear Discriminants are of the linear form:

gⱼ(x)=aⱼ₀+aⱼ₁*x₁+aⱼ₂*x₂+ . . . +aⱼᵢ*xᵢ

where x refers to the vector of biomarker values (x₁, x₂, . . . ) for each sample, the subscript j refers to disease state j, and the subscript i refers to biomarker i.

The initial coefficients aⱼᵢ are set by taking the average of each biomarker i for disease state j, and dividing it by the pooled variance of that biomarker across all disease states:

aⱼᵢ=aveⱼ(j)/varᵢ

For discriminating between two disease states, for instance between those positive for appendicitis versus those negative, the two discriminants may be collapsed into a single equation by subtracting the negative discriminant from the positive descriminant:

g(x)=a₀+d₁*x₁+d₂*x₂+ . . . +dᵢ*xᵢ

where dᵢ is sometimes known as the discriminability, and is defined as

dᵢ=(aveᵢ(+)−aveᵢ(−))/varᵢ

In some embodiments, Logistic regression models are of the form:

$$\text{?} = \frac{\text{?}}{1 + \text{?}} + \text{?}$$

$$\text{?} \text{indicates text missing or illegible when filed}$$

where y′ᵢ=[1, x₁, x₂, . . . xⱼ], and x is the value of biomarker i, β is the vector of model parameters, and the yᵢ are independent Bernoulli random variables (e.g., disease/no disease). The fitted logistic regression model provides an estimate of the probability of disease (or conversely no disease). This probably function can then be used with a cut-off as a diagnostic rule for classifying subjects as positive or negative for disease. See e.g., Montgomery, Peck & Vining (2001) Introduction to Linear Regression Analysis, 3^{rd} ed. John Wiley & Sons, NY, N.Y.; Peduzzi P, Concato J, Kemper E, Holford T R, Feinstein A R (1996). “A simulation study of the number of events per variable in logistic regression analysis,” J Clin Epidemiol 49 (12): 1373-9. PMID 8970487; Agresti A (2007). “Building and applying logistic regression models,” An Introduction to Categorical Data Analysis. Hoboken, N.J.: Wiley. p. 138. ISBN 978-0-471-22618-5; Jonathan Mark and Michael A. Goldberg (2001), Multiple Regression Analysis and Mass Assessment: A Review of the Issues, The Appraisal Journal, January pp. 89-109; Agresti, Alan. (2002), Categorical Data Analysis, New York: Wiley-Interscience. ISBN 0-471-36093-7; Hilbe, Joseph M. (2009), Logistic Regression Models, Chapman & Hall/CRC Press, ISBN 978-1-4200-7575-5; Hosmer, David W.; Stanley Lemeshow (2000), Applied Logistic Regression, 2nd ed., New York; Chichester, Wiley, ISBN 0-471-35632-8.

In some embodiments, principal component analysis can be used. Principal component analysis is a dimension reduction technique that is used to combine multiple variables into a new set of variables, or principal components, which are uncorrelated and ordered such that the first few retain most of the variation present in the original data. In some embodiments, each principal component is of the form:

αⱼ′x=α₁x₁+α₂x₂+ αₖxₖ

where the xᵢ is the value of biomarker i and the αᵢ are the PC scores that are derived from the eigenvectors of XX'. One or more of the principal components can then be used to derive a diagnostic rule. See e.g., Jolliffe (2002) Principal Component Analysis, 2^{nd} ed. Springer, N.Y., N.Y.

The classifier can be optimized by varying the coefficients, using the discriminabilities as the starting point. The Downhill Simplex method can be used to optimize for AUROC. For the purposes of determining a ROC curve, however, all multiples of S(x) are equivalent, so that the coefficients could wander by orders of magnitude during the optimization. Therefore, the last coefficient can be held fixed to keep the function within range. Clearly, any of the coefficients could have been held constant without affecting the final result.

ROC curves are generated by ranking samples according to the value of the discriminant function, and plotting sensitivity parametrically against 1-specificity.³⁴⁻³⁶ Since it is only the relative values of the discriminant function that matter, linear discriminants that yield identical ROC curves (and thus identical AUROC) can return values that vary dramatically. The optimization process could therefore return discriminant functions which did not verify well, as the verification process comprises or consists of training the discriminant functions against bootstrapped sample sets. Therefore, each optimization can be finished by multiplying the discriminant function by a factor which would set the value at which the sensitivity equaled the specificity to 0.5.

Any suitable reference value can be used in the present methods. For example, the reference value can be a threshold value or a reference range. In some embodiments, the threshold value can be obtained from a population with appendicitis, a population without appendicitis, a population cured or recovered from appendicitis, or a subject before having appendicitis, having appendicitis, cured or recovered from appendicitis. In other embodiments, the reference range can be obtained from a population with appendicitis, a population without appendicitis, a population cured or recovered from appendicitis, or a subject before having appendicitis, having appendicitis, cured or recovered from appendicitis. The following Table 2 shows exemplary normal reference range of the selected biomarkers.

TABLE 2
Exemplary normal reference range of the selected biomarkers

| Biomarker | Normal reference range | |
|---|---|---|
| MRP 8/14 | 0.05-2.0 | μg/ml |
| CRP | 0.3-6.0 | μg/ml |
| A1AT | 1.5-3.5 | mg/ml |
| SAA 1 | 4.0 | μg/ml |
| MMP-1 | 533 | ng/ml |
| MDH-1 | 10-1000 | ng/ml |
| TIMP-1 | 133 | ng/ml |
| PCT | 10-100 | ng/ml |
| MMP-9 | 45-60 | ng/ml |
| HA | 37 | ng/ml |
| IL-6 | 9 | pg/ml |

The present methods can further comprises a step of separating a subject into a group of having high risk for appendicitis or a group of having low risk for appendicitis before determining values of the biomarkers in a sample from the subject. The subjects can be separated into a group of having high or low risk for appendicitis by any suitable ways or standards. For example, the subjects can be separated into a group of having high or low risk for appendicitis by assessing general inflammation level of the subject. The general inflammation level can be assessed by any suitable ways or standards. For example, the general inflammation level of the subject can be assessed by determining values of CRP, SAA and/or IL-6 in a sample from the subject.

In some embodiments, the subjects can be separated into a group of having high or low risk for appendicitis by assessing a physical sign or symptom such as duration of a symptom, duration of abdominal pain, diffuse abdominal pain, focal right lower quadrant (RLQ) abdominal pain, RLQ tenderness, pain with percussion, rebound tenderness, pain with cough, hop, heel tap, difficulty in walking, pain migrated Right iliac fossa/RLQ, history of similar pain, anorexia, nausea, vomiting, temperature, Rovsing's sign, and rigidity or guarding, WBC, ANC and/or percent neutrophil in WBC. Any suitable number of the physical signs or symptoms, e.g., at least 2, 3, 4, 5, or 6 physical signs or symptoms, are assessed.

In some embodiments, the physical signs or symptoms shown in the Table 3 below, or any combination thereof, are used.

TABLE 3
Exemplary physical signs or symptoms

| Shorthand | Symptom | Description of Symptom |
|---|---|---|
| PE1 | Duration of | The length of which the symptoms |
| | symptoms | have been present and/or persisting |
| | | in said patient |
| PE2 | Duration of | The length of which the abdominal |
| | abdominal pain | pain has been present and/or |
| | | persisting in said patient |
| PE3 | Diffuse abdominal | Not definitely limited or |
| | pain | localized abdominal pain |
| PE4 | Focal RLQ | Pain localized to the Right Lower |
| | abdominal pain | Quadrant of the abdomen |
| PE5 | RLQ tenderness | A state of unusual sensitivity |
| | | to touch or pressure, specifically |
| | | localized to the Right Lower |
| | | Quadrant of the abdomen |
| PE6 | Pain with | Percussion: the act of striking a part |
| | percussion | with short, sharp blows as an aid in |
| | | diagnosing the condition of the |
| | | underlying parts by the sound |
| | | obtained |
| PE7 | Rebound | a state in which pain is felt on the |
| | tenderness | release of pressure over a part; |
| | | includes tenderness to percussion; |
| PE8 | Pain w/cough, hop, | Abdominal pain associated with |
| | heel tap | coughing, hopping, or the tapping of |
| | | ones heels |
| PE9 | Difficulty walking | |
| PE10 | Pain migrated Rt. Iliac | Pain originates in the right iliac fossa |
| | Fossa/RLQ | and moves to the right lower quadrant |
| PE11 | History of similiar | |
| | pain | |
| PE12 | Anorexia | Lack or loss of appetite |
| PE13 | Nausea | An unpleasant sensation vaguely |
| | | referred to the epigastrium and |
| | | abdomen, with a tendency to vomit |
| PE14 | Vomiting | Forcible ejection of the contents of the |
| | | stomach through the mouth |
| PE15 | Temperature | An expression of heat or coldness in |
| | | terms of a specific scale |
| PE17a | Rovsing's Sign | Palpation of the lower left quadrant |
| | | of the abdomen results in increased |
| | | pain in the right lower quadrant |
| PE17b | Rigidity or | Rigidity: inflexibility or stiffness-i.e. |
| | guarding | avoidance of motion, continual flexing |
| | | of the hips resulting in the relief of |
| | | tension in the abdominal musculature |
| | | Guarding: |

In other embodiments, the subjects can be separated into a group of having high or low risk for appendicitis by assessing a physical sign or symptom including RLQ tenderness, rebound tenderness, pain migrated right iliac fossa/RLQ, vomiting, rigidity or guarding, and/or Rovsing's sign. At least 2, 3, 4, 5, or 6 physical signs or symptoms are assessed. For example, 6 physical signs or symptoms can be assessed and the subject is separated into a group of having high risk for appendicitis when at least 3 physical signs or symptoms are positive, and the subject is separated into a group of having low risk for appendicitis when less than 3 physical signs or symptoms are positive.

In some embodiments, the physical signs or symptoms are used to generate the exemplary clinical rules for separating subjects into a group of having high or low risk for appendicitis as shown in Table 4 below.

TABLE 4
Exemplary clinical rules

| | PE 5 | PE 7 | PE 10 | PE 14 | PE 17 | |
|---|---|---|---|---|---|---|
| | Tender- | Re- | Migra- | Vom- | Rigidity/ | PE 17 |
| | ness | bound | tion | iting | guarding | Rovsings |
| CPR3.1 | (+) | (+) | (+) | | | |
| CPR3.2 | (+) | (+) | | (+) | | |
| CPR3.3 | (+) | (+) | | | (+) | |
| CPR3.4 | (+) | (+) | | | | (+) |
| CPR3.5 | (+) | | (+) | (+) | | |
| CPR3.6 | (+) | | (+) | | (+) | |
| CPR3.7 | (+) | | | (+) | | (+) |
| CPR3.8 | (+) | | | (+) | (+) | |
| CPR3.9 | (+) | | | (+) | | (+) |
| CPR3.10 | (+) | | | | (+) | (+) |
| CPR3.11 | | (+) | (+) | (+) | | |
| CPR3.12 | | (+) | (+) | | (+) | |
| CPR3.13 | | (+) | (+) | | | (+) |
| CPR3.14 | | (+) | | (+) | (+) | |
| CPR3.15 | | (+) | | (+) | | (+) |
| CPR3.16 | | (+) | | | (+) | (+) |
| CPR3.17 | | | (+) | (+) | (+) | |
| CPR3.18 | | | (+) | (+) | | (+) |
| CPR3.19 | | | (+) | | (+) | (+) |
| CPR3.20 | | | | (+) | (+) | (+) |

In some embodiments, the methods having a step of separating a subject into a group of having high risk for appendicitis or a group of having low risk for appendicitis before determining values of the biomarkers in a sample from the subject have enhanced performance compared to a corresponding method wherein the subjects have not been separated into a group of having high risk for appendicitis or a group of having low risk for appendicitis. The performance enhancement can be any suitable aspect of the method, e.g., the assay sensitivity, specificity, positive predictive value and/or negative predictive value. For example, some exemplary assays have an AUC value of at least 0.7, 0.75, 0.8, 0.85, 0.9 or higher. Some exemplary assays have an assay sensitivity value of at least 0.8, 0.85, 0.9, 0.91, 0.92, 0.93, 0.94, 0.95, 0.96, 0.97, 0.98, 0.99, or higher. Some exemplary assays have an assay specificity value of at least 0.3, 0.4, 0.5, 0.6, 0.7, 0.8, 0.9 or higher. Some exemplary assays have a negative predictive value of at least 0.8, 0.85, 0.9, 0.91, 0.92, 0.93, 0.94, 0.95, 0.96, 0.97, 0.98, 0.99, or higher. Some exemplary assays have some or all of an AUC value, an assay sensitivity value, an assay specificity value, positive predictive value and a negative predictive value as described above.

The present methods can be used for any suitable purposes. For example, the present methods can be used for diagnosis, prognosis, stratification, risk assessment, or treatment monitoring of appendicitis in a subject. In another example, the present methods can be used for ruling out appendicitis from a subject in a low risk group. In still another example, a subject is diagnosed, prognosed of having appendicitis, or deemed to have increased risk of appendicitis when the test score from the subject is higher than a reference value, e.g., a reference value obtained from a population without appendicitis, a population with appendicitis, a population cured or recovered from appendicitis, or the same subject before having appendicitis, having appendicitis, cured or recovered from appendicitis. In yet another example, a subject is diagnosed, prognosed of not having appendicitis, or deemed to have decreased risk of appendicitis when the test score from the subject is substantially the same or lower than a reference value, e.g., a reference value obtained from a population without appendicitis, a population with appendicitis, a population cured or recovered from appendicitis, or the same subject before having appendicitis, having appendicitis, cured or recovered from appendicitis. In yet another example, a subject in a low risk group is ruled out from having appendicitis, or deemed for not needing any further test, when the test score from the subject falls within, or is lower than, a normal reference value of the low risk group.

In another aspect, provided herein is a device or system for assessing appendicitis in a subject, which device or system comprises: a) means for determining values of a plurality of biomarkers in a sample from a subject; and b) a computer readable medium containing executable instructions that when executed combine said values of said biomarkers using a mathematical algorithm to produce a numerical test score.

The present devices can further comprise a computer readable medium containing executable instructions that when executed compare the test score to a reference value to assess appendicitis in a subject, and/or the reference value.

Any suitable means can be used for determining values of a plurality of biomarkers. For example, the means for determining values of a plurality of biomarkers can comprise binding reagents that specifically bind to the biomarkers.

The present methods, devices and/or systems can be further optimized depending on the goal or purpose of the assays. In one example, the present methods, devices and/or systems can be further optimized to enhance or maximize assay sensitivity, often within the desired range of assay specificity. Often in this case, the assay sensitivity is enhanced or maximized so that a negative assay result will have enhanced or maximized negative predictive value, e.g., a negative assay result accurately identifying the subject not having appendicitis. In another example, the present methods, devices and/or systems can be further optimized to enhance or maximize assay specificity, often within the desired range of assay sensitivity. Often in this case, the assay specificity is enhanced or maximized so that a positive assay result will have enhanced or maximized positive predictive value, e.g., a positive assay result accurately identifying the subject having appendicitis.

### C. Methods and Kits for Assessing Appendicitis in a Subject

In another aspect, provided herein is a method for assessing appendicitis in a subject, which method comprises: a) separating a subject into a group of having high risk for appendicitis or a group of having low risk for appendicitis; b) determining values of a plurality of biomarkers in a sample from said subject; and c) comparing said values of said biomarker to a reference value of the corresponding group to assess appendicitis in said subject.

The subject can be separated into a group of having high or low risk for appendicitis by any suitable ways or standards. In some embodiments, the subject can be separated into a group of having high or low risk for appendicitis by assessing general inflammation level of the subject. The general inflammation level of the subject can be assessed by any suitable ways or standards. For example, the general inflammation level of the subject can be assessed by determining value of CRP, SAA and/or IL-6 in a sample from the subject.

In some embodiments, the subject can be separated into a group of having high or low risk for appendicitis by assessing a physical sign or symptom, or any combination thereof, such as duration of a symptom, duration of abdominal pain, diffuse abdominal pain, focal right lower quadrant (RLQ) abdominal pain, RLQ tenderness, pain with percussion, rebound tenderness, pain with cough, hop, heel tap, difficulty in walking, pain migrated right iliac fossa/RLQ, history of similar pain, anorexia, nausea, vomiting, temperature, Rovsing's sign, and rigidity or guarding, WBC, ANC and/or percent neutrophil in WBC. In some embodiments, the subject is separated into a group of having high or low risk for appendicitis by assessing a physical sign or symptom selected from the group consisting of RLQ tenderness, rebound tenderness, pain migrated right iliac fossa/RLQ, vomiting, rigidity or guarding, and Rovsing's sign, or any combination thereof.

Any suitable number of physical signs or symptoms can be assessed. In some embodiments, at least 2, 3, 4, 5, or 6 physical signs or symptoms are assessed. In some embodiments, 6 physical signs or symptoms are assessed and the subject is separated into a group of having high risk for appendicitis when at least 3 physical signs or symptoms are positive, and the subject is separated into a group of having low risk for appendicitis when less than 3 physical signs or symptoms are positive.

In some embodiments, the methods have enhanced performance compared to a corresponding method wherein the subjects have not been separated into a group of having high risk for appendicitis or a group of having low risk for appendicitis. The performance enhancement can be any suitable aspect of the method, e.g., the assay sensitivity, specificity and/or negative predictive value. For example, some exemplary assays have an AUC value of at least 0.7, 0.75, 0.8, 0.85, 0.9 or higher. Some exemplary assays have an assay sensitivity value of at least 0.8, 0.85, 0.9, 0.91, 0.92, 0.93, 0.94, 0.95, 0.96, 0.97, 0.98, 0.99, or higher. Some exemplary assays have an assay specificity value of at least 0.3, 0.4, 0.5, 0.6, 0.7, 0.8, 0.9 or higher. Some exemplary assays have a negative predictive value of at least 0.8, 0.85, 0.9, 0.91, 0.92, 0.93, 0.94, 0.95, 0.96, 0.97, 0.98, 0.99, or higher. Some exemplary assays have an AUC value, an assay sensitivity value, an assay specificity value and a negative predictive value as described above.

The values of the biomarkers can be determined by any suitable ways. For example, the values of the biomarkers can be determined by determining amounts, concentrations and/or activities of the biomarkers.

Any suitable biomarkers can be used in the present methods. In some embodiments, the biomarkers are myeloid related protein 8/14 (MRP 8/14), C-reactive protein (CRP), hyaluronan (HA), matrix metalloproteinase-9 (MMP-9), serum amyloid A1 (SAA 1), serum amyloid A2 (SAA 2), 1-antitrypsin (A1AT), epidermal growth factor (EGF), endothelial-leukocyte adhesion molecule 1 (ELAM-1 or E-Selectin), granulocyte colony-stimulating factor (G-CSF or GCSF), glutathione s-transferase omega-1 (GSTO1), interleukin-6 (IL-6), interleukin-8 (IL-8). junction plakoglobin (JUP), Layilin, lectin, galactose binding, soluble 3 (Lgals3), malate dehydrogenase (MDH or MADH), matrix metalloproteinase-1 (MMP-1), neural cell adhesion molecule 1 (NCAM 1), nuclear factor NF-kappa-B p105 subunit (NFKB-1), plasminogen activator inhibitor-1 (PAI-1), Parkinson disease (autosomal recessive, early onset) 7 (Park-7), procalcitonin (PCT), metallopeptidase inhibitor I (TIMP-1), urokinase-plasminogen activator (UPA), vascular endothelial growth factor D (VEGF-D), white blood cell count (WBC), absolute neutrophil count (ANC) and/or percent neutrophil in WBC (% NEU).

In some embodiments, the biomarker are myeloid related protein 8/14 (MRP 8/14), C-reactive protein (CRP), hyaluronan (HA), matrix metalloproteinase-9 (MMP-9), serum amyloid A1 (SAA 1), serum amyloid A2 (SAA 2), white blood cell count (WBC), absolute neutrophil count (ANC) and/or percent neutrophil in WBC. In some embodiments, the combination of the biomarkers does not include the combination of CRP and WBC, CRP and WBC, CRP and SAA, SAA and WBC, or CRP, SAA and WBC.

The values of at any suitable number of biomarkers can be determined and compared to the corresponding reference value(s). For example, at least 2, 3, 4, 5, 6, 7, 8, 9 or 10 biomarkers can be determined and compared to the corresponding reference value(s).

In some embodiments, the values of the biomarkers can be combined using a mathematical algorithm to produce a numerical test score, and the test score is compared to a reference value to assess appendicitis in the subject. Any suitable mathematical algorithm can be used. For example, the mathematical algorithm can be Naïve Bayesian Classifiers (NBC), Fisher Linear Discriminants (FLD) and/or Logistic Regression (LR).

Any suitable samples can be used in the present methods. For example, the sample can be a serum, a plasma and a blood sample. In another example, the sample can be a clinical sample. The present methods can be used for assessing appendicitis in any suitable subject. For example, the subject can be a human, and the biomarkers are corresponding human biomarkers. In some embodiments, the human subject is a male, female, an infant, a child, a teenager, a young adult, e.g., a young adult, less than 18, 21, 25 or 30 years old, a middle aged person or a senior.

The values of the biomarkers can be determined by any suitable reagents. For example, the values of the biomarkers can be determined using binding reagents that bind to, and preferably specifically bind to, the biomarkers. Exemplary binding reagents include antibodies, receptors, especially soluble receptors, and aptamers. The values of the biomarkers can be determined by any suitable methods. For example, the values of the biomarkers can be determined by an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, surface plasmon resonance (SPR), chemiluminescence assay, lateral flow immunoassay, u-capture assay, inhibition assay or avidity assay.

Any suitable reference value can be used in the present methods. For example, the reference value can be a threshold value or a reference range. In some embodiments, the threshold value can be obtained from a population with appendicitis, a population without appendicitis, a population cured or recovered from appendicitis, or a subject before having appendicitis, having appendicitis, cured or recovered from appendicitis. In other embodiments, the reference range can be obtained from a population with appendicitis, a population without appendicitis, a population cured or recovered from appendicitis, or a subject before having appendicitis, having appendicitis, cured or recovered from appendicitis.

The present methods can be used for any suitable purposes. For example, the present methods can be used for diagnosis, prognosis, stratification, risk assessment, or treatment monitoring of appendicitis in a subject. In another example, the present methods can be used for ruling out appendicitis from a subject in a low risk group. In still another example, a subject is diagnosed, prognosed of having appendicitis, or deemed to have increased risk of appendicitis when the test score from the subject is higher than a reference value, e.g., a reference value obtained from a population without appendicitis, a population with appendicitis, a population cured or recovered from appendicitis, or the same subject before having appendicitis, having appendicitis, cured or recovered from appendicitis. In yet another example, a subject is diagnosed, prognosed of not having appendicitis, or deemed to have decreased risk of appendicitis when the test score from the subject is substantially the same or lower than a reference value, e.g., a reference value obtained from a population without appendicitis, a population with appendicitis, a population cured or recovered from appendicitis, or the same subject before having appendicitis, having appendicitis, cured or recovered from appendicitis. In yet another example, a subject in a low risk group is ruled out from having appendicitis, or deemed for not needing any further test, when the test score from the subject falls within, or is lower than, a normal reference value of the low risk group.

In still another aspect, provided herein is a kit or device for assessing appendicitis in a subject, which kit or device comprises: a) means for assessing appendicitis risk in a subject; and b) means for determining value of a biomarker in a sample from said subject.

Any suitable means for assessing appendicitis risk in a subject can be used. For example, the means for assessing appendicitis risk can comprise means for assessing a physical sign or symptom such as duration of a symptom, duration of abdominal pain, diffuse abdominal pain, focal right lower quadrant (RLQ) abdominal pain, RLQ tenderness, pain with percussion, rebound tenderness, pain with cough, hop, heel tap, difficulty in walking, pain migrated right iliac fossa/RLQ, history of similar pain, anorexia, nausea, vomiting, temperature, Rovsing's sign, and rigidity or guarding, WBC, ANC and/or percent neutrophil in WBC.

Any suitable means for determining value of a biomarker can be used. For example, the means for determining value of a biomarker can comprise a binding reagent that specifically binds to the biomarker. Exemplary binding reagents include antibodies, receptors, especially soluble receptors, and aptamers.

The present methods and/or kits can be further optimized depending on the goal or purpose of the assays. In one example, the present methods and/or kits can be further optimized to enhance or maximize assay sensitivity, often within the desired range of assay specificity. Often in this case, the assay sensitivity is enhanced or maximized so that a negative assay result will have enhanced or maximized negative predictive value, e.g., a negative assay result accurately identifying the subject not having appendicitis. In another example, the present methods and/or kits can be further optimized to enhance or maximize assay specificity, often within the desired range of assay sensitivity. Often in this case, the assay specificity is enhanced or maximized so that a positive assay result will have enhanced or maximized positive predictive value, e.g., a positive assay result accurately identifying the subject having appendicitis.

### D. Exemplary Embodiments

Biomarkers are defined as any measureable biological parameter that correlates with disease state. Classically, biomarkers are proteins whose concentrations in the blood or other bodily fluid rise or fall in response to disease, and thus can be used for diagnosis. Biomarkers may also include biological signs such as temperature, blood pressure, etc., or clinical impressions, such as localized pain, nausea, drowsiness, etc.

For most applications, biomarkers are used to distinguish whether a sample comes from a patient who is positive or negative for a specific disease state. Biomarker values may either rise or fall in response to a disease state. In the case of protein biomarkers, these responses would correspond to “up regulation” or “down regulation”, respectively. Arbitrary threshold values are chosen to decide whether a sample is positive or negative.

Measures of the performance of a biomarker include “sensitivity” and “specificity”. Sensitivity is the fraction of samples which are positive for a disease state which are determined to be positive by the biomarker. Specificity is the fraction of samples which are negative for a disease state which are determined to be negative by the biomarker. Obviously, both of these measures depend on the threshold value that is chosen. Changing the threshold value to increase the sensitivity necessarily decreases the specificity, and vice versa. The Receiver Operator Characteristic Curve (ROC Curve) is a construct in which the sensitivity is plotted parametrically against 1-specificity, for all threshold values.³⁵ The Area Under the ROC Curve (AUROC) is the integrated area under this curve, and summarizes the biomarker performance over all possible threshold values into a single value. The AUROC is a useful metric, since in general a biomarker with a better AUROC will show better performance regardless of what performance metric (sensitivity, specificity, positive or negative predictive value, positive or negative likelihood ratio, etc.) is being used.^{33,34}

Biomarkers may also be combined in order to increase the accuracy of the diagnosis. The mathematical algorithms used to arrive at a decision are known as classifiers, and are a part of an intensely studied branch of applied mathematics known as pattern recognition.^{29,30,37} Pattern recognition consists of analyzing data to deduce which of many possible phenomena produced that data. Many different types of mathematical classifiers have been developed and studied in the literature. These different types of classifiers are known as classifier models, and consist of different ways of combining the data from the multiple biomarkers to arrive at a result. Any of these models may be applied to the same data to arrive at a decision.

The development of a multi-biomarker classifier requires several steps.^{30,37} First, a training set of samples must be acquired. This may be done prospectively, that is, the data from the samples may be acquired, not the samples themselves. The disease state of each sample must be known. Second, the biomarkers to be used in the classifier must be chosen and their values determined. Third, the classifier model must be chosen. Fourth, the parameters of the model must be determined. These parameters may be calculated from analysis of the entire set of samples, or may be optimized according to some predetermined value function. Finally, the classifier must be evaluated according to some predetermined value function, and accepted or rejected.

A multi-biomarker classifier is only expected to show a significant improvement over all its single biomarker components when the biomarkers show some degree of independence.²⁹ Choosing independent biomarkers is most easily achieved by selecting biomarkers that characterize different aspects of a disease. In the case of appendicitis, for instance, biomarkers specific for inflammation would be expected to be independent of biomarkers specific for structural damage.

In the most general case, a classifier uses biomarker data from a particular sample to classify that sample into one of several different disease states. Each different disease state is associated with a “discriminant function” that returns a score as an output from the biomarker data that is input. Comparison of the scores from the different discriminant functions determines how the sample is classified among the different disease states. In most implementations, the sample is classified according to which discriminant function returns the highest score. In some implementations, a sample may be classified according to if a discriminant function returns a score above a certain threshold value, so that a sample may be classified into multiple disease states.

In the case of classifiers developed to discriminate between two exclusive disease states (for example, positive or negative for appendicitis), the two discriminant functions may be collapsed into a single function by subtracting the two original functions. As in the case for single biomarkers, a single value is produced for any particular sample. Different threshold levels may be arbitrarily set to distinguish positive samples from negative samples. For instance, for a sample having biomarker levels of x1, x2, . . . xn, using a threshold level L, and a discriminant function go, then if g(x1, x2, . . . xn)>L, the sample is positive, otherwise it is negative. It is obvious that any algebraic manipulation of the above inequality will yield the same result, and thus is an identical invention.³⁰ For instance, any of the following operations do not change the results of the above inequality, and therefore do not qualify as unique inventions: adding a constant to both sides of the equation; multiplying both sides of the equation by a positive constant; multiplying both sides of the equation by a negative constant and reversing the inequality; subjecting both sides of the equation to a monotonic function such as natural log, exponential, etc.

Characterizing a classifier by a single discriminant function which returns a single value also allows the use of ROC curves. ROC curves allow the performance of multi-biomarker classifier to be compared with single biomarker performance.

In some embodiments, the present invention uses mathematical algorithms to diagnose whether a patient has appendicitis. The choice of biomarker measurements are used as input to these algorithms, and the means of determining the parameters are used in these algorithms.

In some embodiments, the biomarkers used to build classifiers in this invention comprise absolute neutrophil count (ANC), percent neutrophil, white blood cell Count (WBC), C-reactive protein (CRP), MRP 8/14, SAA1 and/or SAA2, hyaluronan, and MMP-9. All possible combinations of five or fewer of these biomarkers were examined during the selection process. The parameters for each classifier were determined by means dependent on the classifier model being used. The values of the parameters found by these means depended on the sample set used for training.

The initial selection criterion was for high values of area under the ROC curve (AUROC). Classifiers that performed well for initial selection were verified using various bootstrap methods, and validated with a sample set that had no overlap with the training set. During verification, classifiers were considered to have performed well when the confidence intervals for sensitivity and specificity were small. During validation, classifiers were considered to have performed well when the sensitivity and specificity for the validation sample set were near to, or better, than the sensitivity and specificity found for the training set.

The classifier models examined were Naïve Bayesian Classifiers (NBC), Fisher Linear Discriminants (FLD), and Logistic Regression (LR) models. All showed similar performance during selection, but the FLD and LR models behaved better during verification.

In some embodiments, a Fisher Linear Discriminant classifier was developed using the biomarkers CRP, MRP-8/14, and WBC. The classifier was trained on a set of sample designated CP11. The form of the discriminant function was:

a1*x1+a2*x2+a3*x3>L

where x1 is the natural log of WBC (cells per ul), x2 is the natural log of CRP concentration in ug/ml, and x3 is the natural log of MRP 8/14 concentration in ug/ml. The coefficient a1 is preferably in the range of 0.32 to 0.38, a2 is preferably in the range of 0.035 to 0.1005, and a3 is preferably in the range of −0.015 to 0.034. Or more preferably, a1 is in the range of 0.33 to 0.37, a2 is in the range of 0.051 to 0.084, and a3 is in the range of −0.003 to 0.0.022. Or most preferably, a1 is 0.34, a2 is 0.073, and a3 is 0.0027. These ranges were found by bootstrapping the sample set 100 times, and finding the average and standard deviations for each coefficients. Ranges were found by adding and subtracting one or two standard deviations from the averages.

When L=0.7072, the sensitivity was 98% and the specificity was 41% when applied to sample set designated as CP-11. Of course, any algebraic manipulation of this discriminant function could be made without changing its utility. For instance, the most preferable embodiment could be multiplied by 2, so that a1 is 0.68, a2 is 0.146, a3 is 0.0054 and L is 1.4144. Using another algebraic manipulation, the discriminant function could also be written as: a1*x1+a2*x2>L−a3*x3

In another embodiment, a Fisher Linear Discriminant classifier was developed using the biomarkers CRP and MRP 8/14. The classifier was trained on a set of sample designated CP11. The form of the discriminant function was:

a1*x1+a2*x2>L

where x1 is the natural log of CRP concentration in ug/ml, and x2 is the natural log of MRP 8/14 concentration in ug/ml. The coefficient a1 is preferably in the range of 0.31 to 0.43, and a2 is preferably in the range of −0.17 to 0.49. Or more preferably, a1 is in the range of 0.34 to 0.40, and a2 is in the range of 0 to 0.33. Or most preferably, a1 is 0.377, and a2 is 0.031. When L=0.02044, the sensitivity was 95% and the specificity was 42% when applied to sample set designated as CP-11.

In yet another embodiment, a Fisher Linear Discriminant classifier was developed using the biomarkers CRP and MRP 8/14. The classifier was trained on a set of sample designated CP11. The form of the discriminant function was:

a1*x1+a2*x2+a3*x3+a4*x4>L

where x1 is the natural log of ANC (cells per ml), x2 is the natural log of WBC (cells per ml), x3 is the natural log of CRP concentration in ug/ml, and x4 is the natural log of MRP 8/14 concentration in ug/ml. The coefficient a1 is preferably in the range of −0.26 to 0.58, a2 is preferably in the range of −0.19 to 0.59, a3 is preferably in the range of 0.029 to 0.12, and a4 is preferably in the range of −0.018 to 0.05. Or more preferably, a1 in the range of −0.05 to 0.37, a2 is in the range of 0.0 to 0.39, a3 is in the range of 0.052 to 0.097, and a4 is in the range of 0.0 to 0.033. Or most preferably, a1 is 0.144, a2 is 0.2057, a3 is 0.0843, and a4 is 0.00187. When L=0.71, the sensitivity was 97% and the specificity was 48% when applied to the sample set designated as CP11.

In another embodiment, a Naïve Bayesian Classifier was developed using the biomarkers CRP, MRP 8/14 and WBC. The form of the discriminant function was:

Ln(p(w)/(1−p(w))<0.02264+0.5*((A−a11)/s11)̂2+0.5*((B−a21)/s21)̂2+0.5*((C−a31)/s31)̂2−0.5*((A−a12)/s12)̂2−0.5*((B−a22)/s22)̂2−0.5*((C+a32)/s32)̂2

where p(w) is an assumed prevalence that varies from 0 to 1, and provides for different levels of sensitivity and specificity, A is the natural log of WBC (cells per ml), B is the natural log of CRP concentration in ug/ml, and C is the natural log of MRP 8/14 concentration in ug/ml. The terms aij refers to the average concentration of biomarker i in disease state j, so that i=1 corresponds to WBC, i=2 corresponds to CRP, and i=3 corresponds to MRP 8/14; and j=1 corresponds to appendicitis, while j=2 corresponds to negative for appendicitis. The terms sij refers to the standard deviation of the concentration of biomarker i in disease state j, so that i=1 corresponds to WBC, i=2 corresponds to CRP, and i=3 corresponds to MRP 8/14; and j=1 corresponds to appendicitis, while j=2 corresponds to negative for appendicitis.

In a most preferred embodiment of the invention, all is 2.569, sl l is 0.3962, a21 is 3.022, s21 is 1.452, a31 is 0.5931, s31 is 0.8121, a12 is 2.115, s12 is 0.4057, a22 is 1.005, s22 is 2.002, a32 is 0.007092, and s32 is 0.8318. When p(w) is set to 0.9323, the sensitivity is 98%, and the specificity is 44%.

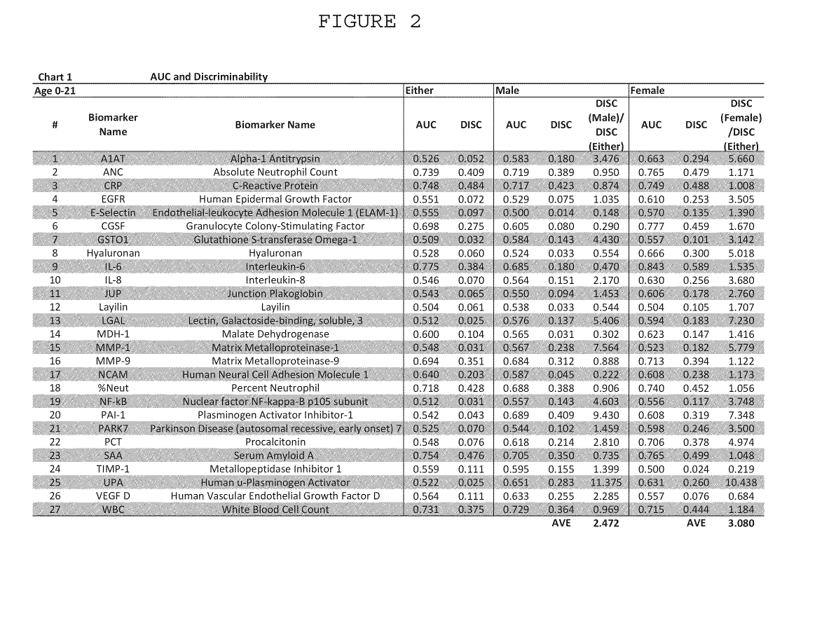

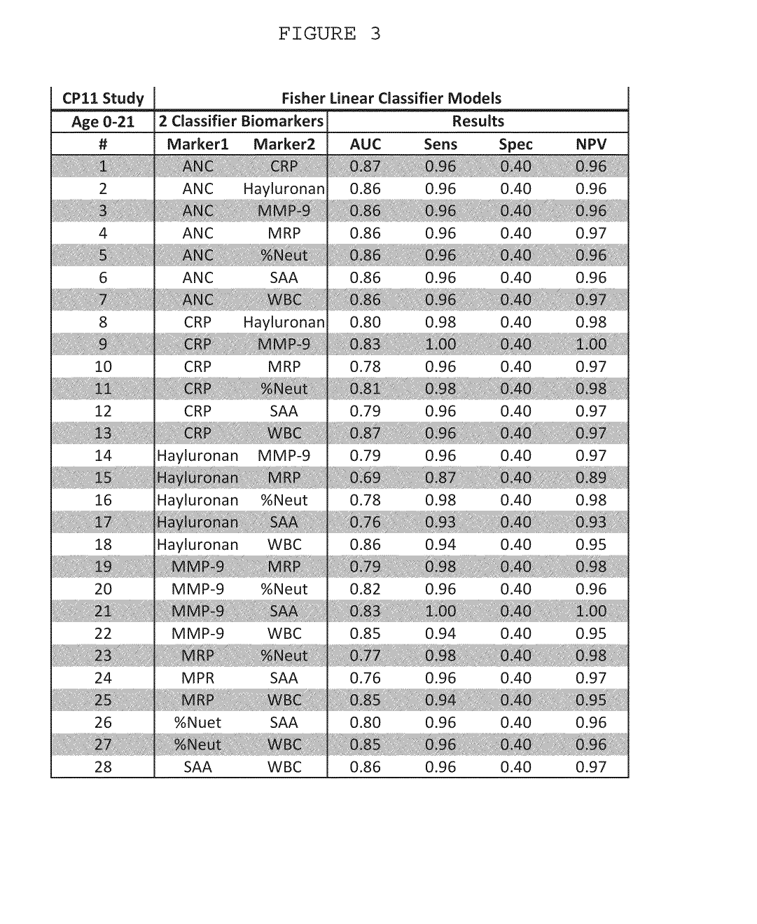

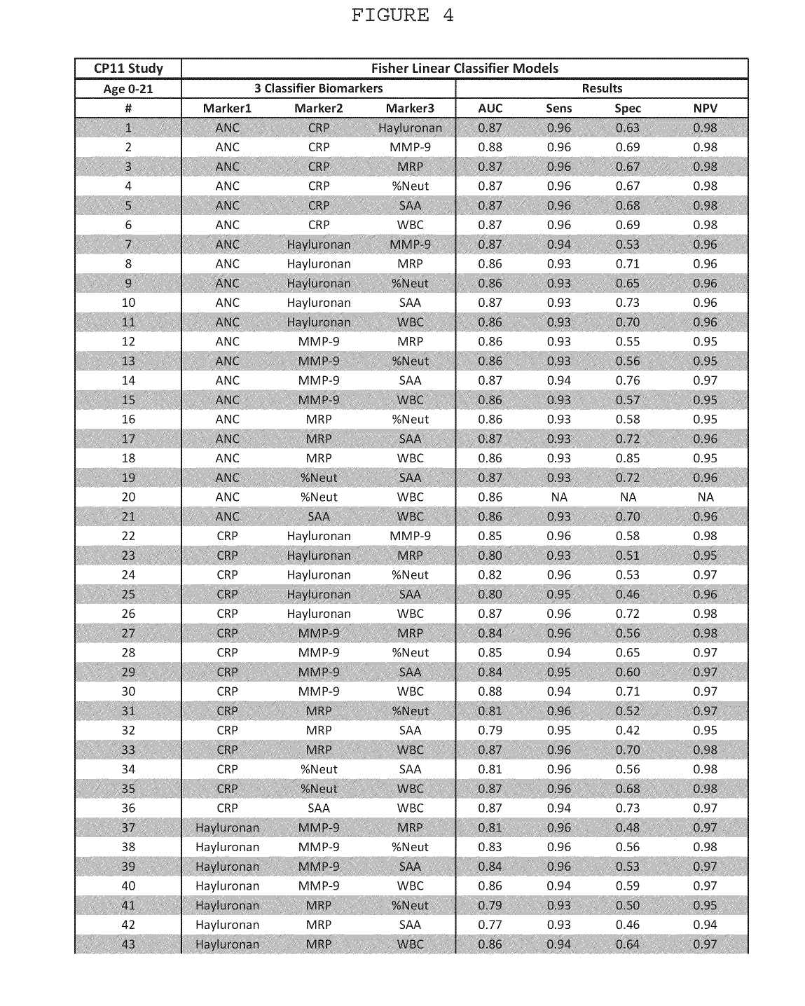

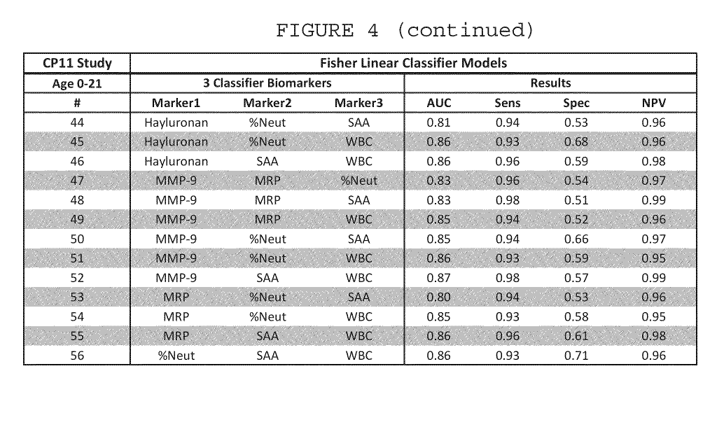

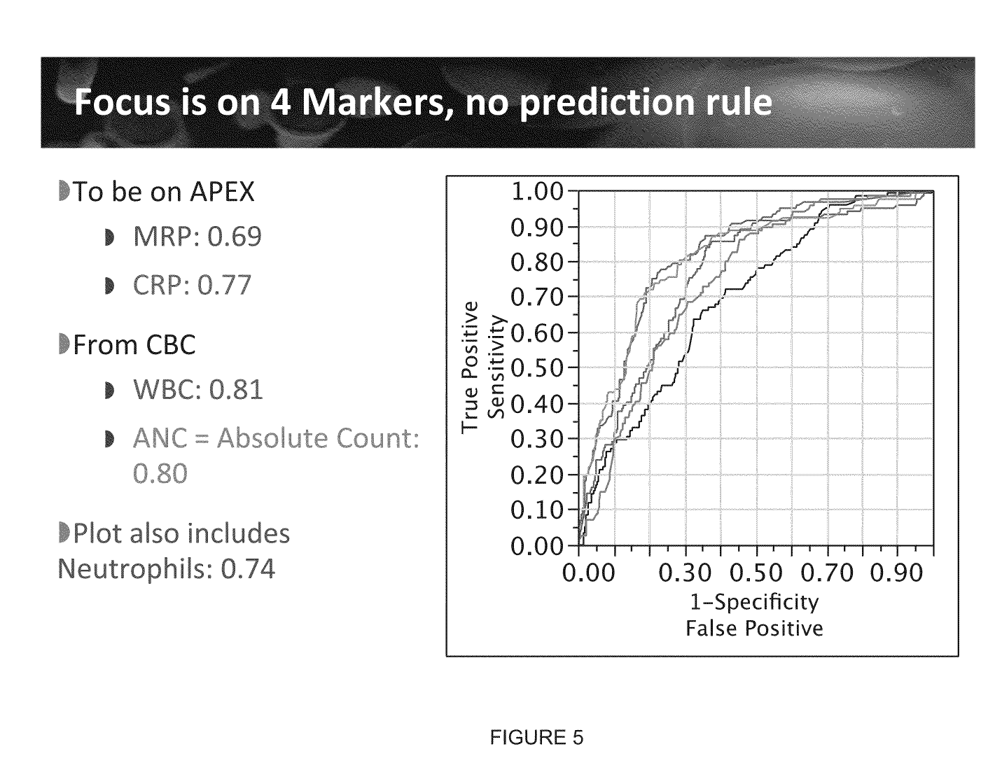

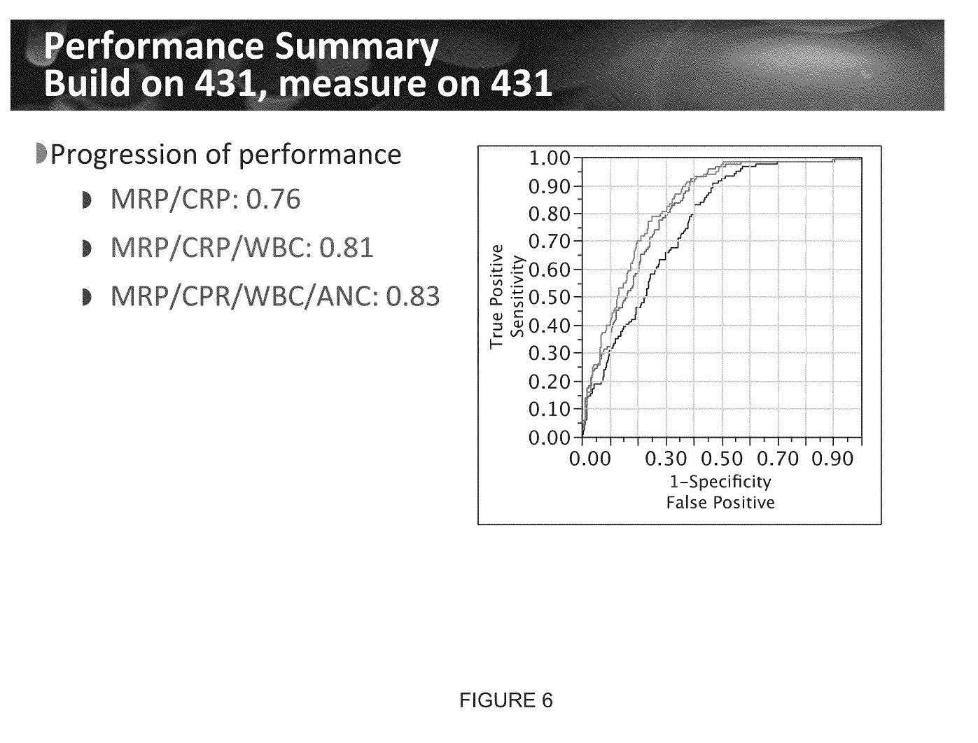

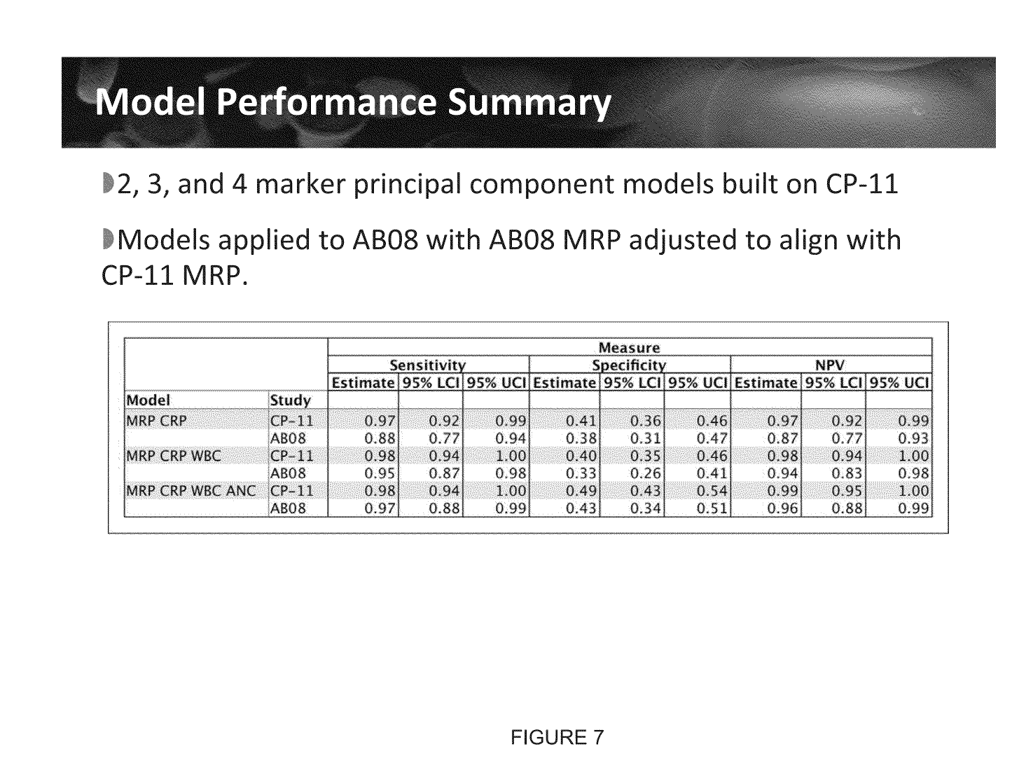

The AUC and discriminability of exemplary biomarkers are shown in FIG. 2. The assay sensitivity, specificity and negative predicable value of exemplary combinations of two biomarkers are shown in FIG. 3. The assay sensitivity, specificity and negative predicable value of exemplary combinations of three biomarkers are shown in FIG. 4. FIG. 5 shows properties of an exemplary four marker (MRP 8/14, CRP, WBC and ANC) combination assay. FIGS. 6 and 7 show properties of exemplary two (MRP 8/14 and CRP), three (MRP 8/14, CRP and WBC) and four (MRP 8/14, CRP, WBC and ANC) marker combination assays.

The present invention is further illustrated by the following exemplary embodiments:

1. A method for assessing appendicitis in a subject, which method comprises:

a) determining values of a plurality of biomarkers in a sample from a subject;

b) combining said values of said biomarkers using a mathematical algorithm to produce a numerical test score; and c) comparing said test score to a reference value to assess appendicitis in said subject.

2. The method of embodiment 1, wherein the values of biomarkers are selected from the group consisting of amounts, concentrations and activities of the biomarkers.

3. The method of embodiment 1 or 2, wherein the biomarkers are selected from the group consisting of myeloid related protein 8/14 (MRP 8/14), C-reactive protein (CRP), hyaluronan (HA), matrix metalloproteinase-9 (MMP-9), serum amyloid A1 (SAA 1), serum amyloid A2 (SAA 2), α1-antitrypsin (A1AT), epidermal growth factor (EGF), endothelial-leukocyte adhesion molecule 1 (ELAM-1 or E-Selectin), granulocyte colony-stimulating factor (G-CSF or GCSF), glutathione s-transferase omega-1 (GSTO1), interleukin-6 interleukin-8 (IL-8), junction plakoglobin (JUP), Layilin, lectin, galactose binding, soluble 3 (Lgals3), malate dehydrogenase (MDH or MADH), matrix metalloproteinase-1 (MMP-1), neural cell adhesion molecule 1 (NCAM 1), nuclear factor NF-kappa-B p105 subunit (NFKB-1), plasminogen activator inhibitor-1 (PAI-1), Parkinson disease (autosomal recessive, early onset) 7 (Park-7), procalcitonin (PCT), metallopeptidase inhibitor 1 (TIMP-1), urokinase-plasminogen activator (UPA), vascular endothelial growth factor D (VEGF-D), white blood cell count (WBC), absolute neutrophil count (ANC) and percent neutrophil in WBC (% NEU).

4. The method of any of the embodiments 1-3, wherein the values of at least 2, 3, 4, 5, 6, 7, 8, 9 or 10 biomarkers are determined and combined into a test score.

5. The method of embodiment 1 or 2, wherein the biomarkers are selected from the group consisting of myeloid related protein 8/14 (MRP 8/14), C-reactive protein (CRP), hyaluronan (HA), matrix metalloproteinase-9 (MMP-9), serum amyloid A1 (SAA 1), serum amyloid A2 (SAA 2), white blood cell count (WBC), absolute neutrophil count (ANC) and percent neutrophil in WBC (% NEU).

6. The method of embodiment 5, wherein the values of at least 2, 3, 4, or 5 of the biomarkers are determined and combined into a test score.

7. The method of any of the embodiments 1-6, wherein the combination of the biomarkers does not include the combination of CRP and WBC, CRP and WBC, CRP and SAA, SAA and WBC, or CRP, SAA and WBC.

8. The method of embodiment 1 or 2, wherein the biomarkers are selected from the group consisting of ANC and CRP; ANC and HA; ANC and MMP-9; ANC and MRP 8/14; ANC and % NEU; ANC and SAA; ANC and WBC; CRP and HA; CRP and MMP-9; CRP and MRP 8/14; CRP and % NEU; CRP and SAA; CRP and WBC; HA and MMP-9; HA and MRP 8/14; HA and % NEU; HA and SAA; HA and WBC; MMP-9 and MRP 8/14; MMP-9 and % NEU; MMP-9 and SAA; MMP-9 and WBC; MRP 8/14 and % NEU; MRP 8/14 and SAA; MRP 8/14 and WBC; % NEU and SAA; % NEU and WBC; SAA and WBC; ANC, CRP and HA; ANC, CRP and MMP-9; ANC, CRP and MRP 8/14; ANC, CRP and % NEU; ANC, CRP and SAA; ANC, CRP and WBC; ANC, HA and MMP-9; ANC, HA and MRP 8/14; ANC, HA and % NEU; ANC, HA and SAA; ANC, HA and WBC; ANC, MMP-9 and MRP 8/14; ANC, MMP-9 and % NEU; ANC, MMP-9 and SAA; ANC, MMP-9 and WBC; ANC, MRP 8/14 and % NEU; ANC, MRP 8/14 and SAA; ANC, MRP 8/14 and WBC; ANC, % NEU and SAA; ANC, % NEU and WBC; ANC, SAA and WBC; CRP, HA and MMP-9; CRP, HA and MRP 8/14; CRP, HA and % NEU; CRP, HA and SAA; CRP, HA and WBC; CRP, MMP-9 and MRP 8/14; CRP, MMP-9 and % NEU; CRP, MMP-9 and SAA; CRP, MMP-9 and WBC; CRP, MRP 8/14 and % NEU; CRP, MRP 8/14 and SAA; CRP, MRP 8/14 and WBC; CRP, % NEU and SAA; CRP, % NEU and WBC; CRP, SAA and WBC; HA, MMP-9 and MRP 8/14; HA, MMP-9 and % NEU; HA, MMP-9 and SAA; HA, MMP-9 and WBC; HA, MRP 8/14 and % NEU; HA, MRP 8/14 and SAA; HA, MRP 8/14 and WBC; HA, % NEU and SAA; HA, % NEU and WBC; HA, SAA and WBC; MMP-9, MRP 8/14 and % NEU; MMP-9, MRP 8/14 and SAA; MMP-9, MRP 8/14 and WBC; MMP-9, % NEU and SAA; MMP-9, % NEU and WBC; MMP-9, SAA and WBC; MRP 8/14, % NEU and SAA; MRP 8/14, % NEU and WBC; MRP 8/14, SAA and WBC; % NEU, SAA and WBC.

9. The method of any of the embodiments 1-8, wherein the sample is selected from the group consisting of a serum, a plasma and a blood sample.

10. The method of any of the embodiments 1-9, wherein the sample is a clinical sample.

11. The method of any of the embodiments 1-10, wherein the values of the biomarkers are determined by a format selected from the group consisting of an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, surface plasmon resonance (SPR), chemiluminescence assay, lateral flow immunoassay, u-capture assay, inhibition assay and avidity assay.

12. The method of any of the embodiments 1-11, wherein the subject is a human.

13. The method of embodiment 12, wherein the human is a child or a young adult less than 30 years old.

14. The method of any of the embodiments 1-13, wherein the mathematical algorithm is selected from the group consisting of Naïve Bayesian Classifiers (NBC), Fisher Linear Discriminants (FLD) and Logistic Regression (LR).

15. The method of any of the embodiments 1-14, wherein the reference value is a threshold value or a reference range.

16. The method of embodiment 15, wherein the threshold value is obtained from a population with appendicitis, a population without appendicitis, a population cured or recovered from appendicitis, or a subject before having appendicitis, having appendicitis, cured or recovered from appendicitis.

17. The method of embodiment 15, wherein the reference range is obtained from a population with appendicitis, a population without appendicitis, a population cured or recovered from appendicitis, or a subject before having appendicitis, having appendicitis, cured or recovered from appendicitis.

18. The method of any of the embodiments 1-17, which further comprises a step of separating a subject into a group of having high risk for appendicitis or a group of having low risk for appendicitis before determining values of the biomarkers in a sample from the subject.

19. The method of embodiment 18, wherein the subject is separated into a group of having high or low risk for appendicitis by assessing general inflammation level of the subject.

20. The method of embodiment 19, wherein the general inflammation level of the subject is assessed by determining values of CRP, SAA and/or IL-6 in a sample from the subject.

21. The method of embodiment 18, wherein the subject is separated into a group of having high or low risk for appendicitis by assessing a physical sign or symptom selected from the group consisting of duration of a symptom, duration of abdominal pain, diffuse abdominal pain, focal right lower quadrant (RLQ) abdominal pain, RLQ tenderness, pain with percussion, rebound tenderness, pain with cough, hop, heel tap, difficulty in walking, pain migrated right iliac fossa/RLQ, history of similar pain, anorexia, nausea, vomiting, temperature, Rovsing's sign, and rigidity or guarding, WBC, ANC and percent neutrophil in WBC.

22. The method of embodiment 18, wherein the subject is separated into a group of having high or low risk for appendicitis by assessing a physical sign or symptom selected from the group consisting of RLQ tenderness, rebound tenderness, pain migrated right iliac fossa/RLQ, vomiting, rigidity or guarding, and Rovsing's sign.

23. The method of embodiment 21 or 22, wherein at least 2, 3, 4, 5, or 6 physical signs or symptoms are assessed.

24. The method of any of the embodiments 18-23, wherein 6 physical signs or symptoms are assessed and the subject is separated into a group of having high risk for appendicitis when at least 3 physical signs or symptoms are positive.

25. The method of any of the embodiments 1-24, which is used for diagnosis, prognosis, stratification, risk assessment, or treatment monitoring of appendicitis in a subject.

26. The method of any of the embodiments 18-24, which is used for ruling out appendicitis from a subject in a low risk group.

27. A device or system for assessing appendicitis in a subject, which device or system comprises:

a) means for determining values of a plurality of biomarkers in a sample from a subject; and

b) a computer readable medium containing executable instructions that when executed combine said values of said biomarkers using a mathematical algorithm to produce a numerical test score.

28. The device or system of embodiment 27, which further comprises a computer readable medium containing executable instructions that when executed compare the test score to a reference value to assess appendicitis in a subject, and/or the reference value.

29. The device or system of embodiment 27, wherein the means for determining values of a plurality of biomarkers comprises binding reagents that specifically bind to the biomarkers.

30. A method for assessing appendicitis in a subject, which method comprises:

a) separating a subject into a group of having high risk for appendicitis or a group of having low risk for appendicitis;

b) determining values of a plurality of biomarkers in a sample from said subject; and

c) comparing said values of said biomarker to a reference value of the corresponding group to assess appendicitis in said subject.

31. The method of embodiment 30, wherein the subject is separated into a group of having high or low risk for appendicitis by assessing general inflammation level of the subject.

32. The method of embodiment 31, wherein the general inflammation level of the subject is assessed by determining value of CRP, SAA and/or IL-6 in a sample from the subject.

33. The method of embodiment 30, wherein the subject is separated into a group of having high or low risk for appendicitis by assessing a physical sign or symptom selected from the group consisting of duration of a symptom, duration of abdominal pain, diffuse abdominal pain, focal right lower quadrant (RLQ) abdominal pain, RLQ tenderness, pain with percussion, rebound tenderness, pain with cough, hop, heel tap, difficulty in walking, pain migrated right iliac fossa/RLQ, history of similar pain, anorexia, nausea, vomiting, temperature, Rovsing's sign, and rigidity or guarding, WBC, ANC and percent neutrophil in WBC.

34. The method of embodiment 30, wherein the subject is separated into a group of having high or low risk for appendicitis by assessing a physical sign or symptom selected from the group consisting of RLQ tenderness, rebound tenderness, pain migrated right iliac fossa/RLQ, vomiting, rigidity or guarding, and Rovsing's sign.

35. The method of embodiment 33 or 34, wherein at least 2, 3, 4, 5, or 6 physical signs or symptoms are assessed.

36. The method of any of the embodiments 30-35, wherein 6 physical signs or symptoms are assessed and the subject is separated into a group of having high risk for appendicitis when at least 3 physical signs or symptoms are positive.

37. The method of embodiment 30, wherein the values of the biomarkers are selected from the group consisting of amount, concentration and activity of the biomarker.

38. The method of any of the embodiments 30-37, wherein the biomarkers are selected from the group consisting of myeloid related protein 8/14 (MRP 8/14), C-reactive protein (CRP), hyaluronan (HA), matrix metalloproteinase-9 (MMP-9), serum amyloid A1 (SAA 1), serum amyloid A2 (SAA 2), α1-antitrypsin (A1AT), epidermal growth factor (EGF), endothelial-leukocyte adhesion molecule 1 (ELAM-1 or E-Selectin), granulocyte colony-stimulating factor (G-CSF or GCSF), glutathione s-transferase omega-1 (GSTO1), interleukin-6 (IL-6), interleukin-8 (IL-8), junction plakoglobin (JUP), Layilin, lectin, galactose binding, soluble 3 (Lgals3), malate dehydrogenase (MDH or MADH), matrix metalloproteinase-1 (MMP-1), neural cell adhesion molecule 1 (NCAM 1), nuclear factor NF-kappa-B p105 subunit (NFKB-1), plasminogen activator inhibitor-1 (PAI-1), Parkinson disease (autosomal recessive, early onset) 7 (Park-7), procalcitonin (PCT), metallopeptidase inhibitor I (TIMP-1), urokinase-plasminogen activator (UPA), vascular endothelial growth factor D (VEGF-D), white blood cell count (WBC), absolute neutrophil count (ANC) and percent neutrophil in WBC (% NEU).

39. The method of any of the embodiments 30-37, wherein the biomarker are selected from the group consisting of myeloid related protein 8/14 (MRP 8/14), C-reactive protein (CRP), hyaluronan (HA), matrix metalloproteinase-9 (MMP-9), serum amyloid A1 (SAA 1), serum amyloid A2 (SAA 2), white blood cell count (WBC), absolute neutrophil count (ANC) and percent neutrophil in WBC.

40. The method of any of the embodiments 30-39, wherein the values of at least 2, 3, 4, 5, 6, 7, 8, 9 or 10 biomarkers are determined and compared to the corresponding reference value(s).

41. The method of embodiment 40, wherein the combination of the biomarkers does not include the combination of CRP and WBC, CRP and WBC, CRP and SAA, SAA and WBC, or CRP, SAA and WBC.

42. The method of embodiment 40 or 41, wherein the values of the biomarkers are combined using a mathematical algorithm to produce a numerical test score, and the test score is compared to a reference value to assess appendicitis in the subject.

43. The method of embodiment 42, wherein the mathematical algorithm is selected from the group consisting of Naïve Bayesian Classifiers (NBC), Fisher Linear Discriminants (FLD) and Logistic Regression (LR).

44. The method of any of the embodiments 30-43, wherein the sample is selected from the group consisting of a serum, a plasma and a blood sample.

45. The method of any of the embodiments 30-44, wherein the sample is a clinical sample.

46. The method of any of the embodiments 30-45, wherein the value of the biomarker is determined by a format selected from the group consisting of an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, surface plasmon resonance (SPR), chemiluminescence assay, lateral flow immunoassay, u-capture assay, inhibition assay and avidity assay.

47. The method of any of the embodiments 30-46, wherein the subject is a human.

48. The method of embodiment 47, wherein the human is a child or a young adult less than 30 years old.

49. The method of any of the embodiments 30-48, wherein the reference value is a threshold value or a reference range.

50. The method of embodiment 49, wherein the threshold value is obtained from a population with appendicitis, a population without appendicitis, a population cured or recovered from appendicitis, or a subject before having appendicitis, having appendicitis, cured or recovered from appendicitis.

51. The method of embodiment 49, wherein the reference range is obtained from a population with appendicitis, a population without appendicitis, a population cured or recovered from appendicitis, or a subject before having appendicitis, having appendicitis, cured or recovered from appendicitis.

52. A kit or device for assessing appendicitis in a subject, which kit or device comprises:

a) means for assessing appendicitis risk in a subject; and

b) means for determining value of a biomarker in a sample from said subject.

53. The kit or device of embodiment 52, wherein the means for assessing appendicitis risk comprises means for assessing a physical sign or symptom selected from the group consisting of duration of a symptom, duration of abdominal pain, diffuse abdominal pain, focal right lower quadrant (RLQ) abdominal pain, RLQ tenderness, pain with percussion, rebound tenderness, pain with cough, hop, heel tap, difficulty in walking, pain migrated right iliac fossa/RLQ, history of similar pain, anorexia, nausea, vomiting, temperature, Rovsing's sign, and rigidity or guarding, WBC, ANC and percent neutrophil in WBC.

54. The kit or device of embodiment 52, wherein the means for determining value of a biomarker comprises a binding reagent that specifically binds to the biomarker.

55. The method of any of the embodiments 18-26, which performance is enhanced compared to a corresponding method wherein the subjects have not been separated into a group of having high risk for appendicitis or a group of having low risk for appendicitis.

56. The method of any of the embodiments 30-51, which performance is enhanced compared to a corresponding method wherein the subjects have not been separated into a group of having high risk for appendicitis or a group of having low risk for appendicitis.

### Example 1

### Summary

Background:

Evaluating children for appendicitis is often difficult and strategies have been sought to improve the precision of the diagnosis. Computed tomography (CT) is often used in the diagnostic workup but there are concerns regarding the exposure to ionizing radiation and risk of subsequent radiation-induced malignancy.

Objectives:

We sought to identify a biomarker panel with sufficient sensitivity to rule out pediatric acute appendicitis without the need for additional imaging modalities.

Methods:

We prospectively enrolled 503 subjects aged two to 20 years presenting in 12 U.S. emergency departments who had abdominal pain and other signs and symptoms suspicious for acute appendicitis within the prior 72 hours. Subjects were assessed for 17 clinical attributes and blood samples were analyzed for CBC, differential, and 5 candidate proteins. Based on discharge diagnosis or post-surgical pathology the cohort exhibited a 28.6% prevalence (144/503 subjects) of appendicitis. Biomarker values were evaluated using principal component, recursive partitioning and logistic regression to select the combination that best discriminated between those subjects with and without disease. A mathematical combination of three inflammation-related markers in a panel comprised of myeloid-related protein 8/14 complex (MRP 8/14), C-reactive protein (CRP) and white blood cell count (WBC) provided optimal discrimination.

Results:

This panel exhibited a sensitivity of 96.5% (95% CI, 92-99%), a specificity of 43.2% (95% CI, 38-48%), a negative predictive value of 96.9% (95% CI, 93-99%), and a negative likelihood ratio of 0.08 (95% CI, 0.03-0.19) for acute appendicitis. Sixty of 185 CT scans (32%) were done for patients with negative biomarker panel results which, if deferred, would have reduced CT utilization by one third at the cost of missing five of 144 (3.5%) patients with appendicitis.

Conclusion:

This panel consisting of WBC, CRP, and MRP 8/14 is highly predictive of the absence of acute appendicitis in this cohort. If these results are confirmed, this panel may be useful in identifying pediatric patients with signs and symptoms suggestive of acute appendicitis who are at low risk and can be followed clinically, potentially sparing them exposure to the ionizing radiation of CT.

### Introduction

Pediatric abdominal pain is a diagnostic challenge, especially when acute appendicitis is in the differential diagnosis⁴²⁻⁴⁴. While acute appendicitis is rare in very young children, it is a common pediatric disease reaching a peak incidence in the second decade of life, with more than 75,000 pediatric cases reported yearly in the United States⁴²⁻⁴⁷. Acute appendicitis is the most common reason for urgent/emergent surgery in the pediatric age group, and missed appendicitis is the second leading cause of malpractice judgments against emergency physicians in patients between the ages of 6 years-17 years^{42,43,45-51.}

Obstacles to the accurate diagnosis of pediatric appendicitis are many. The utility of history and physical examination may be limited in young children who lack the verbal skills to accurately describe their symptoms. As many as 50% of children may not have typical signs and symptoms associated with appendicitis, such as anorexia, migration of pain to the right lower quadrant, or focal right lower quadrant tenderness^{42,43,45,48}. The laboratory evaluation of patients with suspected appendicitis, including white blood cell count (WBC), C-reactive protein (CRP), and other serum markers either alone or in combination can be helpful, but the reported sensitivities and specificities for these markers are highly variable and not independently reliable to accurately exclude or confirm the diagnosis^{42,44,46,48,52-57}. Several clinical scoring systems have also been developed to aid in the diagnosis of appendicitis⁵⁸⁻⁶¹. Most, however, are dependent on laboratory data (WBC or absolute neutrophil count), incorporate clinical items such as migration of pain that may be difficult to determine in younger children, have a wide range of weightings for data points, and can be cumbersome to use in the clinical setting. Studies utilizing these scoring systems have yielded inconsistent results, and hence, they are not routinely used in clinical practice^{42-45,48,62-67}.

The definitive diagnosis of appendicitis has become increasingly dependent on radiologic imaging, including both computed tomography (CT) and ultrasound (US). The American College of Emergency Physicians, American College of Radiology, as well as many authors and institutions recommend using US first for pediatric patients in order to avoid the ionizing radiation exposure of CT^{43,44,45,68}. While US has good reported specificity (88-98%), sensitivity is suboptimal (78-100%)^{44,68-72}. Patients are therefore frequently referred for abdominal CT which has improved test characteristics (sensitivity 92-100%, specificity 87-100%) compared to US^{44,70-76}.

### Importance

The use of CT in the Emergency Department (ED) has increased dramatically in the last decade across all age groups including pediatric patients⁷⁷⁻⁷⁹. Hryhorczuk et. al. reported an increase in CT utilization for pediatric ED patients with abdominal pain from 2% in 1999 to 16% in 2007, while Tsze et al. reported that for pediatric patients ultimately diagnosed with appendicitis, CT utilization increased from 0% in 1996 to 60% in 2006^{80,81}. Menoch et. al, however, have shown that when alternatives exist CT utilization may be limited⁸².

There is increasing recognition of the risks of ionizing radiation exposure related to CT, especially for children^{77,83-89}. The smaller body mass of children results in a larger net radiation dose per kilogram and children have more years of life expectancy to carry the increased risk of radiation-induced malignancy^{76,81}. When exposed to a similar radiation dose, a 1 year old child has an estimated 10-15 times the lifetime risk of developing a malignancy than does a 50 year old adult⁸³. It has also been estimated that a single abdominal CT under the age of 10 can increase the lifetime risk of death from radiation-induced malignancy by 1 in 1,000, and that up to 2% of all cancers in the United States in coming decades may be caused by radiation exposure due to CT scans^{76,80,81,83-86}.

For these reasons, alternative diagnostic strategies are needed.

Goals of this Investigation

We sought to evaluate the diagnostic accuracy of individual plasma biomarkers, combinations of these markers, and markers combined with clinical data to assess whether these biomarkers possess adequate sensitivity and negative predictive value to rule out appendicitis in pediatric and adolescent patients with abdominal pain whose differential diagnosis includes acute appendicitis.

### Materials and Methods:

### Setting

This study was conducted at 12 academic and academically-affiliated community hospital EDs in the United States between April 2011 and November 2011 (see acknowledgements for participating site listing). All sites obtained Institutional Review Board (IRB) approval prior to study initiation and all participating patients provided written consent if greater than 18 years old, or written parental consent and patient assent for those under the age of 18 per hospital specific IRB requirements.

### Study Design

This study was a prospective, double blind observational investigation of clinical parameters and plasma protein markers in pediatric and adolescent patients with signs and symptoms suggesting possible acute appendicitis to select classifiers useful in predicting the absence of the disease. Patients who provided consent/assent had blood samples drawn for plasma testing of protein biomarkers with clinical, laboratory, imaging, and treatment data collected for each patient. All patients were enrolled based on presenting symptoms prior to any diagnostic testing or imaging, and decisions regarding evaluation, laboratory testing, imaging, and treatment were determined solely by the treating physician independent of participation in the study.

### Selection of Patients

The study population is a convenience sample of pediatric patients presenting to participating EDs with abdominal pain suggesting possible acute appendicitis. Inclusion criteria were right lower quadrant or generalized abdominal pain with other signs and symptoms suspicious for or consistent with acute appendicitis, duration of symptoms ≦72 hours, and ages from two to twenty years inclusive. Subjects were excluded if they had a history of previous appendectomy, metastatic cancer, bleeding disorder, or active autoimmune disorder. Patients were also excluded if they had abdominal trauma, invasive abdominal procedures, diagnostic imaging for abdominal pain, or participation in other research protocols within the prior two weeks, or if the legally responsible parent or guardian was unwilling or unable to provide consent. Patients were approached sequentially by study staff based on presenting symptoms during times of staff availability. Those who fit inclusion criteria and agreed to participate were enrolled prospectively prior to any diagnostic testing.

### Outcome Measures

The primary outcome measures were the presence or absence of acute appendicitis, defined by surgical pathology report for those who had appendectomy and discharge diagnosis for those who did not have appendectomy, and results of the plasma protein biomarkers. The primary study endpoint was the diagnostic accuracy of the protein biomarkers alone and in combination as a negative predictor for acute appendicitis. Secondary outcome measures and endpoints were the utilization rate of CT scanning and the potential reduction of unnecessary CT scans for those with negative biomarker results should the biomarkers provide adequate diagnostic accuracy for the absence of acute appendicitis.

### Data Collection and Processing

Whole blood samples were drawn into EDTA tubes from consenting subjects and twice centrifuged within two hours at 1300 g, with plasma collected and frozen at −70° C. or colder. Plasma samples were then transported frozen to a single testing laboratory for analysis. Thawed EDTA plasma samples were tested for five protein biomarkers including myeloid-related protein 8/14 complex (MRP 8/14), C-reactive protein (CRP), hyaluronan, serum amyloid A protein (SAA), and matrix metallopeptidase 9 (MMP-9). MRP 8/14 concentrations were determined using an MRP 8/14 lateral flow assay developed by AspenBio Pharma, Inc. (Castle Rock, Colo.). Plasma CRP levels were measured using Siemens' (Tarrytown, N.Y.) Immulite® 1000 hsCRP assay. The plasma MMP-9 and hyaluronan levels were measured in ELISA format using R&D Systems' (Minneapolis, Minn.) DuoSets. SAA was also measured in ELISA format using a goat anti-SAA polyclonal antibody (sc-20275) and a mouse anti-SAA monoclonal antibody (sc-52887) from Santa Cruz Biotechnology Inc. (Santa Cruz, Calif.). The admission complete blood count (CBC) with differential for each subject was obtained from each site's clinical laboratory and recorded.

Standardized case report forms (CRFs) were used to collect demographic, clinical, laboratory, imaging, and treatment data across all practice sites. Clinical data collected included the treating physician's clinical impression (PCI) of the likelihood of appendicitis on a 100 point visual analogue scale prior to any lab or imaging results, and 17 clinical data points including reported duration of illness, symptoms, signs, and physical findings. Information regarding past medical history, comorbid conditions, and preceding treatment with antipyretics was also collected. Timing and results of abdominal ultrasound and/or CT imaging, surgical pathology reports for those who had appendectomy, disposition and discharge diagnosis for all patients, and return visits within 72 hours were also recorded. Data points not noted by the treating clinician were collected by study personnel. In compliance with ICH GCP, study site staff transcribed all source data onto protocol CRFs. CRFs were verified for accuracy by independent study monitors and transmitted to the sponsor for statistical analysis.

Laboratory personnel performing the plasma protein analysis were blinded to the patient's clinical information and outcome, and protein biomarker results were not available to the treating physicians, study staff, or investigators at clinical study sites.

### Primary Data Analysis

The biomarker panel and associated algorithm was derived after in-depth post-hoc analyses of markers and CBC values. Numerous model types, including logistic regression, partitioning, Fisher's linear discriminant, and principal component analysis were used to explore the data to find the best combination of markers to rule out appendicitis. The principal component analysis consistently had slightly better results in terms of diagnostic performance than the other models considered, and hence, was the choice for the panel. The biomarker panel algorithm is the first principal component of WBC, CRP, and MRP 8/14, which is a linear equation combining these three markers into one single value. This single value was calculated for each subject and then compared across the two populations, those with appendicitis and those without appendicitis, to arrive at an optimal cut-off point and subject classification based on the biomarker panel.

### Results

### Patient Population

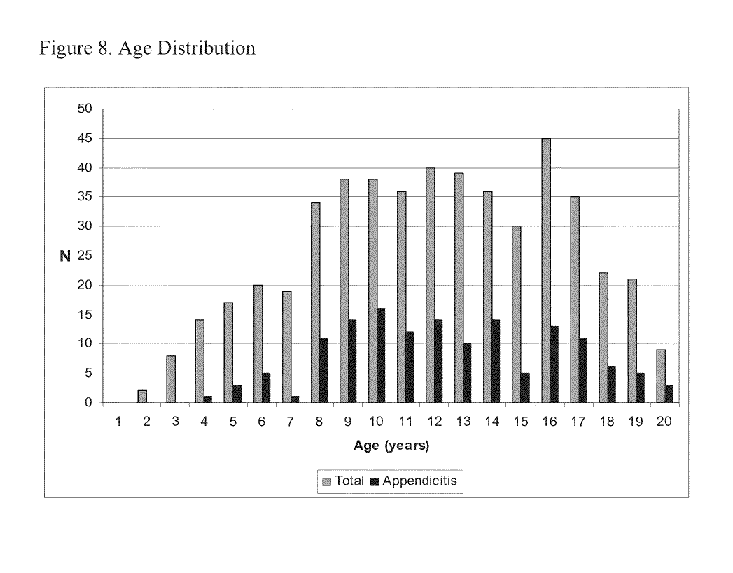

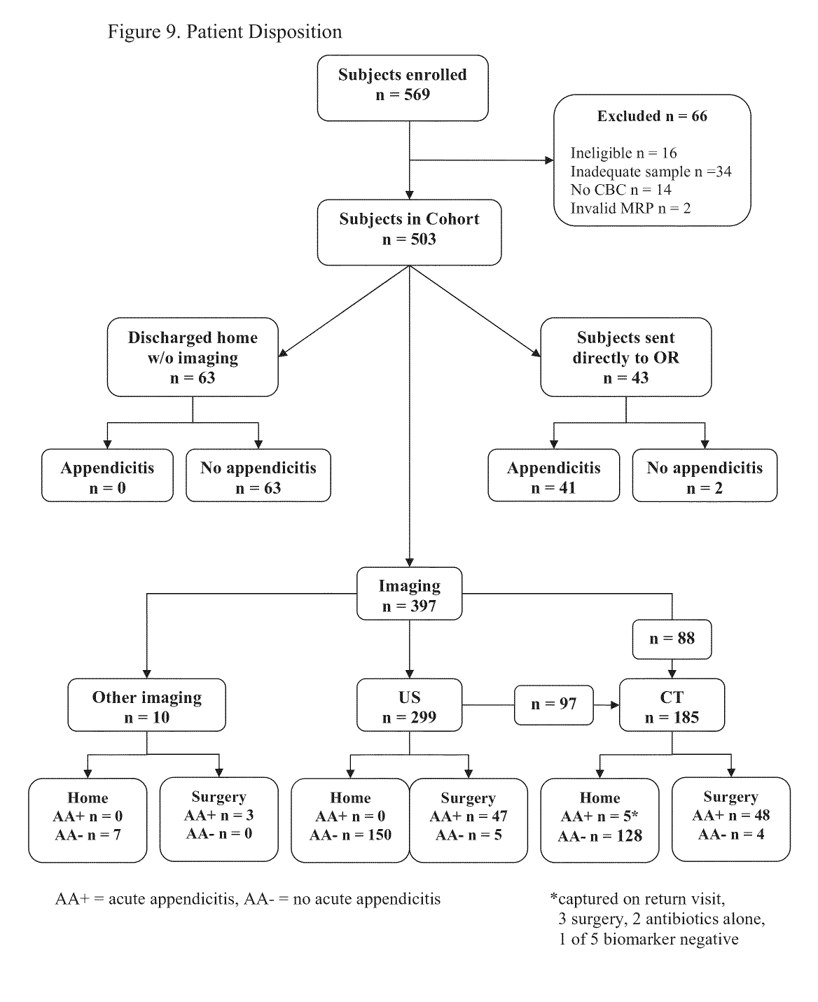

From April 2011 through November 2011 pediatric patients presenting to the EDs of 12 participating institutions with symptoms suggesting acute appendicitis as part of their differential diagnosis were approached for consent to participate. There were 569 patients enrolled, of whom 66 were excluded for ineligibility, lack of adequate samples, or invalid test results, leaving 503 patients in the study cohort. The overall prevalence of appendicitis in the cohort was 28.6% (144/503), with a prevalence of 36% in male patients (87/243) and 22% in female patients (57/260). This difference was statistically significant (p=0.0008). Patient demographics and characteristics are displayed in Table 5, age distribution of patients in FIG. 8, and further data regarding enrolling institution, prevalence of appendicitis, and imaging utilization are displayed in Table 6. The diagnostic pathways including imaging, treatment, and disposition of patients are displayed in FIG. 9.

TABLE 5
Patient demographics and clinical characteristics

| Patient | Appendicitis | Not Appendicitis | |
|---|---|---|---|
| Characteristics | n (n/N %) | n (n/N %) | p-value |
| Total N = 503 | N = 144 (28.6%) | N = 359 (71.4%) | |
| Age, y | | | |
| Median (IQR) | 12 (10-16) | 12 (8-16) | |
| ≦12 years | 77 (53%) | 189 (53%) | 0.9214 |
| Gender | | | |
| Male | 87 (60%) | 156 (43%) | 0.0008 |
| Ethnicity | | | |
| White | 90 (63%) | 247 (69%) | |
| Black | 10 (7%) | 30 (8%) | |
| Hispanic | 32 (22%) | 64 (18%) | 0.3510 |
| Asian | 4 (3%) | 3 (1%) | c² test |
| Other/Unreported | 8 (5%) | 15 (4%) | |
| Symptom duration | | | |
| 0-12 hours | 34 (24%) | 102 (28%) | |
| 12-24 hours | 55 (38%) | 122 (34%) | 0.6676 |
| 24-48 hours | 31 (22%) | 72 (20%) | c² test |
| 48-72 hours | 24 (17%) | 63 (18%) | |
| Similar abdominal pain | | | |
| previously | | | |
| Yes | 14 (10%) | 60 (17%) | 0.0511 |
| Associated symptoms | | | |
| Periumbilical pain with | 100 (69%) | 141 (39%) | <0.0001 |
| migration to RLQ | | | |
| Anorexia | 105 (73%) | 198 (55%) | 0.0003 |
| Vomiting | 92 (64%) | 132 (38%) | <0.0001 |
| Nausea | 109 (76%) | 225 (63%) | 0.0065 |
| Physical examination | | | |
| Fever(≧99.6 F., 37.5 C.) | 36 (25%) | 59 (16%) | 0.320 |
| RLQ Tenderness | 143 (99%) | 309 (86%) | <0.0001 |
| Rebound tenderness | 73 (53%) | 73 (21%) | <0.0001 |
| Rigidity and Guarding | 102/137 (73%) | 110/343 (32%) | <0.0001 |
| Rovsing's sign | 49/137 (36%) | 38/340 (11%) | <0.0001 |
| Imaging | | | |
| US | 68 (47%) | 231 (64%) | 0.0006 |
| CT | 53 (37%) | 132 (37%) | 1.0 |
| Both US and CT | 21 (15%) | 76 (21%) | 0.1042 |
| No imaging | 41 (28%) | 65 (18%) | 0.0113 |
| Other | 3 (2%) | 7 (2%) | *n too |
| | | | small |
| Alternative Diagnoses | | | |
| Abdominal pain | | 186 (53%) | |
| Constipation | | 29 (9%) | |
| Ovarian cyst | | 27 (8%) | |
| Gastroenteritis | | 25 (7%) | |
| UTI | | 13 (4%) | |
| Colitis | | 4 (1%) | |
| Perforated appendix by | n^{1/}/n (%) | | |
| symptom duration (n¹) | | | |
| 0-12 hours | 1/34 (3%) | | |
| 12-24 hours | 0/55 (0%) | | |
| 24-48 hours | 6/31 (19%) | | |
| 48-72 hours | 6/24 (25%) | | |

p-values are based on Fisher's exact test unless otherwise noted

TABLE 6
Hospital type, prevalence of appendicitis, imaging utilization*

| Hospital | | Appendicitis % | CT % | US % | CT and US % |
|---|---|---|---|---|---|
| type | N | (n/N) | (n/N) | (n/N) | (n/N) |
| All | 503 | 28.6% (144/503) | 36.8% (185/503) | 59.4% (299/503) | 19.3% (97/503) |
| Hospitals | | | | | |
| Childrens | 216 | 32.9% (71/216) | 27.8% (60/216) | 45.4% (98/216) | 10.2% (22/216) |
| Hospitals | | | | | |
| Tertiary | 135 | 26.7% (36/135) | 39.3% (53/135) | 79.3% (107/135) | 28.1% (38/135) |
| Care Centers | | | | | |
| Community | 152 | 24.3% (37/152) | 47.4% (72/152) | 61.8% (94/152) | 24.3% (37/152) |
| Hospitals | | | | | |

*imaging totals >100% as those with both CT and US also included in individual totals for CT, US

### Main Results

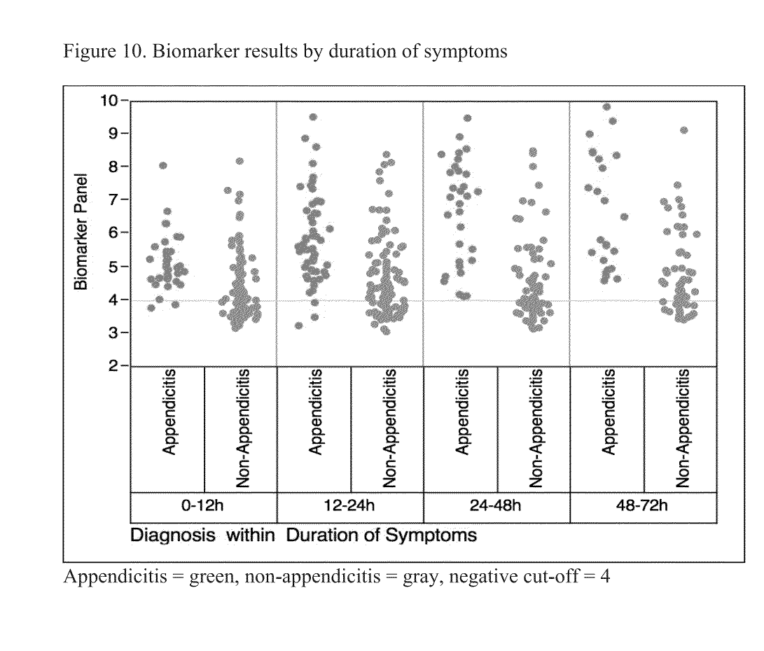

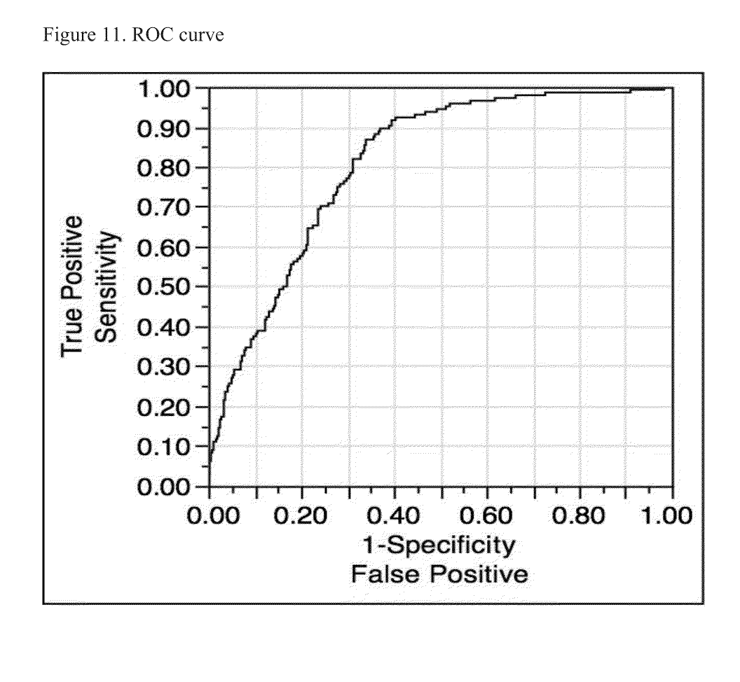

Analysis of the biomarker values using principal components resulted in a best fit mathematical model composed of WBC, CRP, and MRP 8/14 with each of these values treated as a separate continuous variable in a linear equation resulting in a single composite value (A). This equation is mathematically expressed as x(WBC)+y(CRP)+z(MRP 8/14)+k=A. Comparing the composite values in positive and negative subjects, a cut-off was selected for clinical utility maximizing the number of true negative test results while minimizing the number of false negative test results. This cut-off was set near the 4^{th} percentile of the distribution of scores for positive subjects. The composite values exhibited an observed range of greater than three and less than ten, with a negative cut-off of four as shown in FIG. 10. Using this cut-off, 160 of 503 (31.8%) patients had negative biomarker results with 155 true negatives and 5 false negatives as shown in Table 7. The sensitivity for acute appendicitis was 96.5% (95% CI, 92-99%), the specificity 43.2% (95% CI, 38-48%), the negative predictive value 96.9% (95% CI, 93-99%), and the negative likelihood ratio 0.08 (95% CI, 0.03-0.19), displayed in Table 4. The AUC for the ROC curve was 0.81 (FIG. 11).

TABLE 7
Biomarker panel results

| | Biomarker panel | Biomarker panel | |
|---|---|---|---|
| | negative | positive | Total |
| No | 155 | 204 | 359 |
| Appendicitis | | | |
| Acute | 5 | 139 | 144 |
| appendicitis | | | |
| Total | 160 | 343 | 503 |

The diagnostic characteristics of the panel were also examined based on duration of symptoms. The distribution of biomarker results by duration of symptoms is also displayed in FIG. 10. When patients were separated into subgroups by duration of symptoms, there were 313 patients with symptoms ≦24 hours, and 190 patients with symptoms >24 hours. The sensitivity and negative predictive values for patients with symptoms ≦24 hours were 94.4% (95% CI 88-98%) and 95.1% (95% CI 89-98%) respectively, and for patients with symptoms >24 hours, 100% (95% CI 94-100) and 100% (95% CI 94-100), as shown in Table 8.

TABLE 8
Biomarker panel diagnostic characteristics

| Duration of | Sensitivity | Specificity | NPV | NLR |
|---|---|---|---|---|
| symptoms (n) | (95% CI) | (95% CI) | (95% CI) | (95% CI) |
| All patients (503) | 96.5% (92-99%) | 43.2% (38-48%) | 96.9% (93-99%) | 0.08 (0.03-0.19) |
| ≦24 hours (313) | 94.4% (88-98%) | 43.3% (37-50%) | 95.1% (89-98%) | 0.13 (0.06-0.31) |
| >24 hours (190) | 100% (94-100%) | 43.0% (35-51%) | 100% (94-100%) | 0 |

NPV = negative predictive value; NLR = negative likelihood ratio

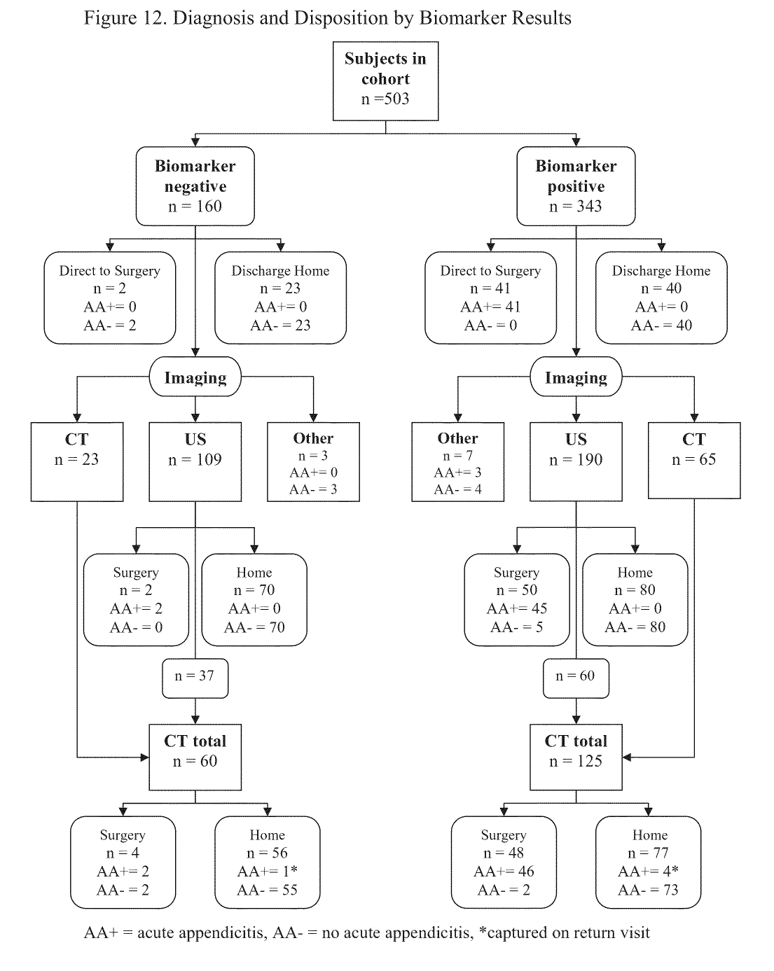

CT scans were performed for 36.8% of patients (185/503). Sixty of the 185 CT scans (32.4%) were performed for patients with negative biomarker panel results as displayed in FIG. 12.

The observed performance of the biomarker panel was then verified by testing against a pediatric subset of 201 subjects drawn from an independent cohort of all ages enrolled in an earlier study. In this cohort, the panel exhibited a sensitivity of 95% (95% CI, 87-98%), a specificity of 36% (95% CI, 29-50%), a negative predictive value of 94% (95% CI, 85-98%), and a negative likelihood ratio of 0.14 (95% CI 0.04-0.42).

### Limitations

This study has several limitations. The greatest limitation is the lack of formal follow up for those patients discharged home from the ED with a diagnosis other than acute appendicitis. The hospital records of all enrolled patients were reviewed for return visits within 72 hours, such that any discharged patient who returned to the same institution with ongoing symptoms would be captured. Five patients with an initial missed diagnosis of acute appendicitis were identified in this manner. However, patients who might have gone to a different hospital for ongoing symptoms would not be detected in this data set, and the prevalence of appendicitis in those patients is unknown. Because of the small number of patients with acute appendicitis and false negative biomarker results, a single missed false negative patient could have a significant effect on the results of the study.

This cohort is a convenience sample, and the differences between patients who were available for consent and chose to participate versus those who did not is a potential source of error. The study population is also disproportionately represented by specialty hospitals for children and academic tertiary care institutions where the prevalence of appendicitis was higher and the imaging rate lower than at community hospitals (Table 6). Results may therefore not be generalizable. However, this suggests that if the test were administered in a larger population with a greater representation of community hospitals the overall prevalence of appendicitis might be lower, thereby increasing the negative predictive value which increases as the prevalence of disease decreases. Based on our data overall imaging rates were also higher at community hospitals, representing an even greater potential for reduction in the utilization of both CT and US. While the subsequent use of CT by those returning within 72 hours with worsening condition was not determined, we were able to estimate the potential decreased use of CT at the time of initial presentation.

Lastly, the plasma samples tested for the protein biomarkers were frozen and shipped to a single laboratory remote from the enrolling hospitals rather than on site using fresh plasma samples as would be done in clinical practice.

### Discussion

In this study of pediatric and adolescent patients with abdominal pain consistent with appendicitis, the three marker panel of WBC, CRP, and MRP 8/14 showed a high sensitivity, high negative predictive value, and low negative likelihood ratio for acute appendicitis. The specificity was low to moderate corresponding to a prevalence of acute appendicitis of 43% in those patients above the negative cut-off value. If these results are confirmed, this panel could have significant value utilized clinically at the bedside as a negative predictor of acute appendicitis in pediatric patients and reduce unnecessary exposure to ionizing radiation. The decision to pursue further imaging, surgical consultation, or appendectomy for patients with values above the negative cut-off should be based on clinical assessment.

Patients in this cohort with negative biomarker panel results represented 32% of all CT scans performed (60/185). If these patients were followed clinically rather than referred for imaging, this would have resulted in a reduction in CT utilization and the associated risks of radiation exposure by one third at initial presentation. However, some patients with negative biomarker results might still be referred for imaging if the treating clinician has a high clinical suspicion or is considering other pathology in addition to acute appendicitis. Patients might also be referred for CT if their condition worsens or remains unclear over time. This estimate is therefore likely the upper range of the potential reduction in CT utilization, but nonetheless demonstrates an opportunity to reduce or limit unnecessary radiation exposure in a subset of patients.

There are additional drawbacks to the reliance on imaging studies beyond radiation exposure. Intravenous (IV) contrast dye poses the risk allergic reactions and renal injury. The cost associated with the increasing utilization of advanced radiologic imaging has been documented to be a major contributor to the rapid rate of medical inflation^{90,91}. Imaging studies also prolong patient evaluation time and ED length of stay^{92,93}. An abdominal CT with oral contrast can add 2-3 hours to a patient's ED stay in order to administer adequate oral contrast. While US and CT with IV, rectal, or no contrast cause less significant delays, all have a negative impact on ED flow and efficiency. In this era of rising ED volumes, the efficiency of diagnosis and disposition is an increasingly important issue.

Ultrasound (US) utilization in this study was even greater than CT. While there is no associated risk of radiation exposure or risk of IV contrast, US can be a significant contributor to the cost of care and length of stay in the ED⁹⁰⁻⁹³. We were not able to determine the number of US in this study done primarily to rule out appendicitis versus those done seeking other diagnostic entities, but the potential impact of this biomarker panel on the utilization of all types of radiologic imaging should not be overlooked.

There were five patients with acute appendicitis who had false negative biomarker results (3.5% of those with appendicitis). Their characteristics are displayed in Table 9. It is worth noting that all these patients had duration of symptoms less than 24 hours, including two with symptoms less than 12 hours. The significance of this observation is that those few patients with false negative biomarker results were still in the early phase of the disease. The outcome to be avoided, appendiceal rupture prior to surgery, is uncommon in the first 24-48 hours^{45,94,95}. In this study the rate of rupture was 1.1% (1/89) for patients with symptoms less than 24 hours and 0% in patients with negative biomarker results. If patients with negative biomarker results and less than 24 hours of symptoms are observed either in the hospital or carefully at home with planned follow up in 12-24 hours, those with continuing or worsening symptoms could then be referred for further testing, imaging, or surgery as appropriate. Based on the data available, negative biomarker panel results in patients with symptoms for more than 24 hours excluded acute appendicitis (Table 8). However, these results must be considered preliminary due of the lack of definitive follow up for patients discharged to home and will require prospective confirmation that includes individual follow up of all patients.

TABLE 9
Patient Characteristics with False Negative Biomarker Results

| | | Duration sx at | | | PCI | | |
|---|---|---|---|---|---|---|---|
| Pt. | Age/sex | enrollment (h) | US | CT | VAS | Clinical Course | Pathology Results |
| A | 16/F | 0-12 h | P | P | 47 | Surgery at index visit | “mild acute appendicitis” |
| B | 12/M | 12-24 h | ND* | N* | 100* | Discharged, returned in | “mild mucosal acute |
| | | | | | | 48 h, CT neg. Surgery | appendicitis” |
| | | | | | | after sx duration 60-72 h | |
| C | 14/F | 12-24 h | I | ND | 67 | Surgery at index visit | “mild acute appendicitis” |
| D | 13/M | 12-24 h | P | ND | 37 | Surgery at index visit | “appendicitis” |
| E | 8/M | 0-12 h | I | P | 23 | Surgery at index visit | “acute appendicitis and |
| | | | | | | | periappendicitis” |

Pt = patient; sx = symptoms; h = hours; P = positive; N = negative; ND = Not done; I = inconclusive; PCI VAS = Physician Clinical Impression Visual Analogue Scale 0-100;
*at index visit

Might this biomarker panel also have an impact on the negative appendectomy rate? In this study, the negative appendectomy rate was 7.2% (11/153), within the range reported in the modern era of CT scanning^{76,96-98}. Four of these 11 patients had negative biomarker results including three with symptoms for less than 24 hours. Two of these three patients went directly to surgery without imaging and one went to surgery after a false positive CT scan. The fourth patient had symptoms for more than 48 hours and went to surgery despite a negative CT scan. If imaging and surgery had been deferred for these four patients in favor of clinical observation, the negative appendectomy rate could have been reduced by 36% (4/11), and the overall negative appendectomy rate could have been 4.7% (7/149) rather than 7.2% (11/153). These results need to be confirmed by further study due to low numbers.

The disparity in the prevalence of appendicitis between male and female patients in this study also deserves mention, although this is not unexpected and is consistent with previous studies¹⁵. Right lower quadrant pain in female patients may be caused by urinary tract infection, ovarian cysts, and other ovarian pathology that are either uncommon or not relevant to male patients. These diseases dilute the prevalence of appendicitis in female patients such that male patients with right lower quadrant pain have a proportionately higher probability of appendicitis.

The strengths of this study are the prospective design, the relatively large number of patients, the broad institutional representation, and the selection of patients based solely on initial presenting symptoms, thereby avoiding any bias that might be introduced by preceding diagnostic evaluation. The study was also blinded such that the results of the test under study did not influence treatments decisions, outcome measures, or endpoints. The test results are objective and quantifiable and, if accurate, should be reproducible and verifiable with further study.

In summary, this study of plasma biomarkers in pediatric and adolescent patients with symptoms suggestive of acute appendicitis produced a three marker panel consisting of WBC, CRP, and MRP 8/14 with high sensitivity, high negative predictive value, and low negative likelihood ratio for acute appendicitis. If patients with negative biomarker panel results had imaging deferred, this could have resulted in a 32% reduction in CT utilization at the time of initial encounter with a potential for decreased overall CT utilization. If these results are confirmed, this panel may be useful in identifying pediatric patients with signs and symptoms suggestive of possible acute appendicitis who are at low risk, are safe for close clinical follow-up, and may be spared unnecessary exposure to ionizing radiation.

### Example 2

### Establishment of AppyScore Cut-Off

Based on data obtained from 503 subjects, ages 2 through 20, in a research clinical study (CP-11), algorithms combining multiple biomarkers were developed to derive a single clinical appendicitis risk measure. Methods explored included; logistic regression, partitioning, discriminate models and principal component analysis (PCA). Performance was evaluated by area under the ROC curve (AUROC) and diagnostic performance (Sp, Se, NPV) comparing to the clinical outcome of confirmed appendicitis as the gold standard measure. The algorithm providing the optimal ROC and diagnostic performance was selected. The PCA method provided the best performance based on the pre-clinical data collected. Ten-fold cross-validation was used to verify the algorithm performance. In addition, the algorithm was verified using results from a retest of the CP-11 samples on kits manufactured in accordance with the Investigational AppyScore IVDMIA system. The resulting, best-performing algorithm is:

AppyScore=0.1177*WBC SI Unit+0.0202*[CRP]μg/ml+1.6*[MRP]μg/ml+(C) Cutoff=4.0 with negatives being a score of ≦4, Observed Range=3−9, C is a constant ranging from about 2 to about 3

In some embodiments, C is 2.4372 or 2.5372.

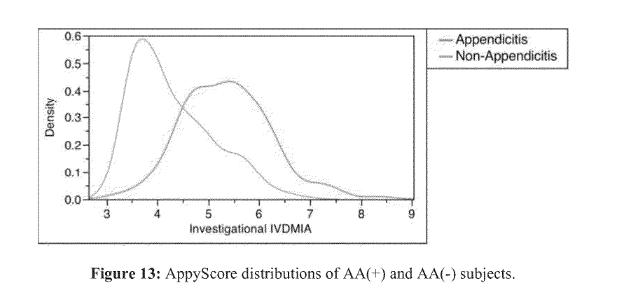

The cut-off for the AppyScore was based on medical feedback and the clinical implications of a negative AppyScore test result within the intended use population. The distributions of AppyScores for Acute appendicitis (AA)(+) and AA(−) subjects overlap, as shown in FIG. 13. It is this ‘mixed clinical presentation’ that makes diagnosis of AA difficult. The optimal AppyScore cut-off for clinical utility will maximize the number of True Negative test results while minimizing the number of False Negative test results. After discussions with clinical experts, it was deemed appropriate to set the cut-off near the 4^{th} percentile of the distribution of scores for positive subjects. This level of False Negatives was deemed acceptable medically.

### Description of Cohort Used to Train the Algorithm on

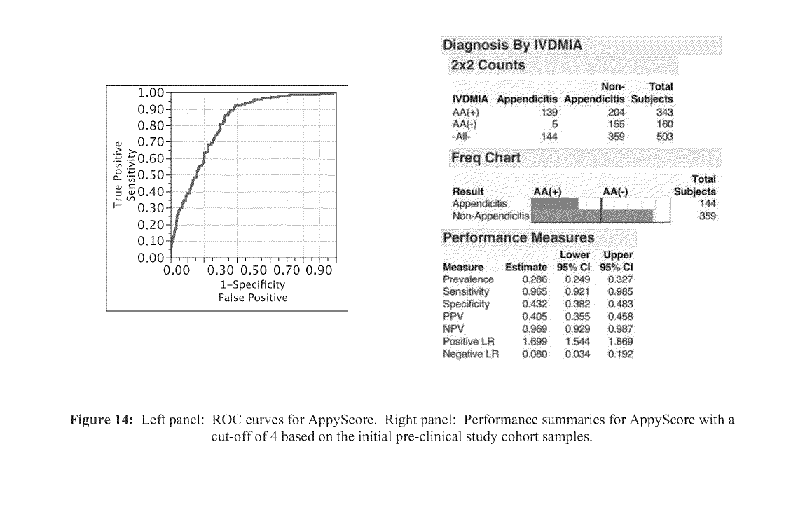

The algorithm was trained on the CP-11 research study cohort, which was the initial evaluation conducted under all the normal IRB and good clinical practice standards. The cohort consisted of 503 pediatric subjects ages 2-20 years presenting in 12 emergency departments with abdominal pain and other signs and symptoms suspicious for acute appendicitis within the prior 72 hours. Performance measures for this cohort are provided in FIG. 14. These data have been used to establish the cut-off for the upcoming pivotal clinical trial.

### B REFERENCE LIST

1. NHAMCS Data Summary for Abdominal Pain and Appendicitis Diagnosis: www.cdc.gov/nchs/ahcd_questionnaires.htm#public_use.
2. Kachualia A, Gandhi T K, Puopolo A L et. al. Missed and delayed diagnoses in the emergency department: a study of closed malpractice claims from 4 liability insurers. An Emer Med 49(1): 196-204. 2007.
3. Paulson E K, Kalady M F, Pappas T N. Suspected appendicitis. N Engl J Med 348(3): 236-242. 2003.
4. Bundy D G, Byerley J S, Liles E A et al. Does this child have appendicitis. JAMA 298(4):438-451. 2007.
5. Howell J M, Eddy O L, Lukens T W et al. Clinical Policy: Critical issues in the evaluation and management of emergency department patients with suspected appendicitis. Ann Emerg Med. 55(1):71-116. 2010.
6. Hennelly K E, Bachur R. Appendicitis Update. Current Opin Pediatri 23(000-000): 1-5. 2011.
7. Wan M J, Krahn M, Ungar W J. Acute appendicitis in young children: Cost effectiveness of US versus CT in diagnosis—a Markov decision analytic model. Radiol. 250(2): 378-386. 2009.
8. Rosendahl k, aukland sm, fosse k. imaging strategies in children with suspected appendicitis. Eur Rad 14(L):138-145. 2004.
9. Selbst S M, Friedman M J, Singh S B. Epidemiology and etiology of malpractice lawsuits involving children in us emergency departments and urgent care centers. Ped Emerg Care. 21(3): 165-169. 2005.
10. Brown T W, McCarthy M L, Kelen G D et al. An epidemiologic study of closed emergency department malpractice claims in a national database of physician malpractice insurers. Soc Acad Emerg Med. 10(1111): 553-560. 2010.
11. Larsen D B, Johnson L W, Schnell B M, et al. Rising Use of CT in child visits to the emergency department in the United States, 1995-2008. Radiol. Published online before print Apr. 5, 2011, doi: 10.1148/radio1.11101939.
12. Bhuiya F, Pitts S R, McCaig L F. Emergency department visits for chest pain and abdominal pain: United States, 1999-2008. NCHC data brief, no 43. Hyattsville, Md.: National Center for Health Statistics 2010.
13. Pines J M. Trends in the rates of radiography use and important diagnoses in emergency department patients with abdominal pain. Med Care 47(7): 782-786. 2009.
14. National Digestive Disease Clearinghouse and other web-based sources is. Bhulya F A, Pitts S A, et al. Emergency department visits for chest pain and abdominal pain: United States, 1999-2008. The National Center for Health Statistics. 43(September): 2010.
16. Glatter R. What role do imaging studies play in diagnosing pediatric appendicitis in the ED? MedScape Emergency Medicine, Web MD. Jun. 18, 2010.
17. Stoker J, Randen A V, et. al. Imaging patients with acute abdominal pain. Radio. 253(1): 31-46. 2009.
18. Rothrock. Acute Appendicitis in Children: Emergency Department Diagnosis and Management. Ann Emerg Med. 36(1): 39-51. 2000
19. Understanding Your Complete Blood Count. Clinical Center National Institutes of Health
20. Alvarado A. A practical score for the early diagnosis of acute appendicitis. An Emerg Med. 1986; 15:557-564.
21. Macklin C P, Merei J M, Radcliffe G S, Stringer M D. A prospective evaluation of the modified Alvarado score for acute appendicitis in children. Ann R Coll Surg Engl. 1997; 79:203-205.
22. Fenyo G. Routine use of a scoring system for decision-making in suspected acute appendicitis in adults. Acta Chir Scand. 1987; 153:545-551.
23. Dado G, Anania G, Baccarani U, et al. Application of a clinical score for the diagnosis of acute appendicitis in childhood: a retrospective analysis of 197 patients. J Pediatr Surg. 2000; 35:1320-1322.
24. Samuel M. Pediatric Appendicitis Score. J Pediatr Surg. 2002; 37:877-881.
25. Schneider C, Kharbanda A, Bachur R. Evaluating appendicitis scoring systems using a prospective pediatric cohort. Ann Emerg Med. 2007; 49:778-784.
26. Bhatt M, Joseph L, Ducharme F M, Dougherty G, McGillivray D. Prospective validation of the pediatric appendicitis score in a Canadian pediatric emergency department. 2009; 16:591-596.
27. Lintula H, Pesonen E, Kokki H, Vanamo K, Eskelinen M. A diagnostic score for children with suspected appendicitis. Langenbecks Arch Surg. 2005; 390:164-170.
28. Sack U, Biereder B, Elouahidi T, et al. Diagnostic value of blood inflammatory markers for detection of acute appendicitis in children. BMC Surg. 2006; 6:15.
29. Evolution of Clinical Proteomics and its Role in Medicine. Boja, Emily, et al. 2010, Journal of Proteome Research.
30. Duda, Richard O, Hart, Peter E and Stork, David G. Pattern Classification. s.l.: John Wiley & Sons, 2001. ISBN 0-471-05669
31. Development and Validation of Biomarker Classifiers for Treatment Selection. Simon, Richard. 2, s.l.: NIH Public Access, 2008, J Stat Plan Inference, Vol. 138, pp. 308-320.
32. Unlocking Biomarker Discovery: Large Scale Application of Aptamer Proteomic Technology for Early Detection of Lung Cancer. Ostroff, R M, et al. 12, 2010, PLoS ONE, Vol. 5, p. e15003. Somalogic 1.
33. Beyond Independence: Conditions for the Optimality of the Simple Bayesian Classifier. Domingos, Pedro and Pazzani, Michael. 1997, Machine Learning, Vol. 29, pp. 103-130.
34. The use of receiver operating characteristic curves in biomedical informatics. Lasko, Thomas A, et al. s.l.: Elsevier, 2005, Journal of Biomedical Informatics, Vol. 38, pp. 404-415.
35. Receiver-Operating Characteristic (ROC) Plots: A Fundamental Evaluation Tool in Clinical Medicine. Zweig, Mark H and Campbell, Gregory. 4, 1993, Clinical Chemistry, Vol. 39, pp. 561-577.
36. Fawcett, Tom. ROC Graphs: Notes and Practical Considerations for Researchers. HP Laboratories. Palo Alto: s.n., 2004. MS1143.
37. Marsland, Stephen. Machine Learning. An Algorithmic Perspective. Boca Raton, Fla.: CRC Press, 2009. ISBN 978-1-4200-6718-7.
38. Larson, Roland E, Hostetler, Robert P and Edwards, Bruce H. Multivariable Calculus. Lexington Mass.: D.C. Heath and Company, 1994. ISBN 0-669-39345-2.
39. Prediction error estimation: a comparison of resampling methods. Molinaro, Annette M, Simon, Richard and Pfeiffer, Ruth M. 15, 2005, Bioinformatics, Vol. 21, pp. 3301-3307.
40. A Paradigm for Class Prediction Using Gene Expression Profiles. Radmacher, Michael D, McShane, Lisa M and Simon, Richard. 3, 2002, Journal of Computational Biology, Vol. 9, pp. 505-511.
41. Pivotol Evaluation of the Accuracy of a Biomarker Used for Classification or Prediction: Standards for Study Design. Pepe, S Margaret, et al. 2008, J Natl Cancer Inst, Vol. 100, pp. 1432-1438.
42. Bundy D G, Byerly J S, Liles E A, Perrin E M, Katznelson J, Rice H E. Does this child have appendicitis? JAMA 2007; 298 (4): 438-451.
43. Hennelly K, Bachur R. Appendicitis update. Current Opinion Pediatrics. 2011; 23: 1-5.
44. Howell J M, Lukens O L, Thiessen M E, et al. Clinical policy: critical issues in the evaluation and management of emergency department patients with suspected appendicitis. Ann Emerg Med 2010; 55: 71-116.
45. Vissers R J, Lennarz W B. Pitfalls in appendicitis. Emerg Med Clin N Am 2010; 28: 103-118.
46. Guthery S L, Hutchings C, Dean J M, Hoff C. National estimates of hospital utilization by children with gastrointestinal disorders: analysis of the 1997 kids inpatient database. J Pediatr 2004; 144(5): 589-594.
47. Hall M J, DeFrances C J, Williams S N, Golosinskiy A, Schwartzman A. National Hospital Discharge Survey: 2007 summary. National health statistics reports; no. 29. Hyattsville, Md.: National Center for Health Statistics. 2010.
48. Rothrock S G, Pagane J. Acute appendicitis in children: emergency department diagnosis and management. Ann Emerg Med 2000; 36 (1): 39-51.
49. Selbst S M, Friedman M J, Singh S B. Epidemiology and etiology of malpractice lawsuits involving children in US emergency departments and urgent care centers. Pediatr Emerg Care 2005; 21: 165-169.
50. Kachalia A, et al. Missed and delayed diagnoses in the emergency department: A study of closed malpractice claims from 4 liability insurers. Ann Emerg Med 2007; 49(2): 196-204.
51. Brown T, et al. An epidemiological study of closed emergency department malpractice claims in a national database of physician malpractice insurers. Acad Emerg Med 2010; 17(5): 553-560.
52. Peltola H, Ahlqvist J, Rapola J, et al. C-reactive protein compared with white blood cell count and erythrocyte sedimentation rate in the diagnosis of acute appendicitis in children, Acta Chir Scand 1986; 152: 55-58.
53. Gronroos J M., Gronroos P. Leukocyte count and C-reactive protein in the diagnosis of acute appendicitis. Br J Surg 1999; 86: 501-504.
54. Anderson R E B. Meta-analysis of the clinical and laboratory diagnosis of appendicitis. Br J Surg 2004; 91: 28-37.
55. Bealer J F, Colgin M. S100A8/A9: A potential new diagnostic aid for acute appendicitis. Acad Emer Med 2010; 17 (3): 333-336.
56. Mills A M, Huckins D S, Kwok H, Baumann B M, et al. Diagnostic Characteristics of S100A8/A9 in a multicenter study of patients with acute right lower quadrant abdominal pain. Acad Emerg Med 2012; 19: 48-55.
57. Kharbanda A, et al. Novel serum and urine markers for pediatric appendicitis. Acad Emerg Med 2012; 19: 56-62.
58. Alvarado A. A practical score for the early diagnosis of acute appendicitis. Ann Em Med 1986; 15 (5): 557-564.
59. Samuel M. Pediatric appendicitis score. J of Pediatr Surg 2002; 37 (6): 877-881.
60. Kharbanda A B, Taylor G A, Fishman S J, Bachur R G. A clinical decision rule to identify children at low risk for appendicitis. Pediatr 2005; 116 (3): 709-716.
61. Andersson M, Andersson R E. The appendicitis inflammatory response score: a tool for the diagnosis of acute appendicitis that outperforms the Alvarado score. World J Surg 2008; 32: 1843-1849.
62. Gwynn L K. The diagnosis of acute appendicitis: clinical assessment versus computed tomography evaluation. J Emerg Med 2001; 21: 119-123.
63. Schneider C, Kharbanda A, Bachur R. Evaluating appendicitis scoring systems using a prospective pediatric cohort. Ann Emerg Med 2007; 49 (6): 778-784.
64. Goldman R D, Carter S, Stephens D, Antoon R, Mountstephen W, Langer J. Prospective validation of the pediatric appendicitis score. J Pediatr 2008; (v): 278-282.
65. Yildrim E, Karagluie E, Kirbas I. Alvarado scores and pain onset in relation to multislice CT findings in acute appendicitis. Diag Interv Radiol 2008: 14; 14-18.
66. Bhatt M, Joseph L, Ducharme F M, Dougherty G, McGillivray D. Prospective validation of the pediatric appendicitis score in a Canadian pediatric emergency department. Acad Emerg Med 2009; 16 (7): 591-596.
67. Unpublished data, AspenBio Pharma, Inc. survey results, 2012.
68. American College of Radiology, Practice Guidelines and Technical Standards, Right lower quadrant pain—suspected appendicitis. Available online at http://www.acr.org/Search?q=appendicitis. Accessed Jun. 9, 2012.
69. Baldisserotto M, Marchiori E. Accuracy of noncompressive sonography of children with appendicitis according to the potential positions of the appendix. Am J Roentgenol 2000; 175: 1387-1392.
70. Sivi C J, Applegate K E, Stallion A, et al. Imaging evaluation of suspected appendicitis in a pediatric population: effectiveness of sonography versus CT. Am J Roentgenol 2000; 175: 977-980.
71. Lowe L H, Penney M W, Stein S M, et al. Unenhanced limited CT of the abdomen in the diagnosis of appendicitis in children: comparison with sonography. Am J Reontgenol 2001; 175: 977-980.
72. Kaiser S, Frenckner B, Jorulf H K. Suspected appenditcitis in children: US and CT-a prospective randomized study. Radiology 2002; 223: 633-638.
73. Acosta R, Crain E F, Goldman H S. CT can reduce hospitalization for observation in children with suspected appendicitis. Pediatr Radiol 2005; 35: 495-500.
74. Dorai A S, Moineddin R, Kellenberger C J, et al. US or CT for diagnosis of appendicitis in children and adults? A meta-analysis. Radiology 2006; 24(1): 83-94.
75. Kharbanda A B, Taylor G A, Bachur R G. Suspected appendicitis in children: rectal and intravenous contrast-enhanced versus intravenous contrast-enhanced CT. Radiology 2007; 243: 530-526.
76. Pickhardt P J, Lawrence E M, Pooler D, Bruce R J. Diagnostic performance of multidetector computed tomography for suspected acute appendicitis. Ann Int Med 2011; 154(12): 789-796.
77. Brenner D, Hall E. Computed tomography—an increasing source of radiation exposure. N Engl J Med 2007; 357: 2277-2284.
78. Kocher K E, Meurer W J, Fazel R, Scott P A, et al. National trends in the use of computed tomography in the emergency department. Ann Emerg Med 2011; 58: 452-562.
79. Larson et al. Rising use of CT in child visits to the emergency department in the United States, 1995-2008. Radiology 2011; 259(3): 793-801.
80. Hryhorczuk A L, Mannix R C, Taylor G A. Pediatric abdominal pain: use of imaging in the emergency department in the United States from 1999-2007. Radiology 2012; doi:10.1148/radio1.12111726.
81. Tsze D S, Asnis L M, Merchant R C, Amanullah S, et al. Increasing computed tomography use for patients with appendicitis and discrepancies in pain management between adults and children: analysis of the NHAMCS. Ann Emerg Med 2012; 59(5): 395-403.
82. Menoch M J, Hirsh D A, Khan N S, Simon H K, Sturm J J, MD. Pediatric emergency department CT trends: changes during increased radiation risk awareness, 2003-2010. Pediatrics 2012; 129(3): e690-7. doi:10.1542/peds.2011-2548.
83. Brenner D, Elliston C, Hall E, et al. Estimated risks of radiation-induced fatal cancer from pediatric CT. AJR Am J Roentgenol 2001; 176: 289-296.
84. Brody A S, Frush D P, Huda W, et al. Radiation risk to children from computed tomography. Pediatrics 2007; 120: 677-682.
85. Picano E. Sustainability medical imaging. BMJ 2004328:578-580.
86. Barrett T W, Schriger D W. Computed tomography imaging in the emergency department: benefits, risks and risk ratios: answers to the November 2011 journal club questions. Am Emerg Med 2012; 59(4): 328-334.
87. Berrington de González A, Mahesh M, Kim K, et al. Projected cancer risks from computed tomographic scans performed in the United States in 2007; Arch Int Med 2009; 169(22): 2071-2077.
88. Smith-Bindman R, et al. Radiation dose associated with common computed tomography examinations and the associated lifetime attributable risk of cancer. Arch Int Med 2009; 169(22): 2078-2086.
89. Pearce M S, Salottti J A, Little M P, McHugh K, et al. Radiation exposure from CT scans in childhood and subsequent risk of leukaemia and brain tumours: a retrospective cohort study. Lancet 2012; doi: 10.1016/50140-6736(12)60815-0.
90. Bodenheimer T. High and rising health care costs. Part 2: technologic innovation. Ann Int Med 2005; 142: 932-937.
91. Hillman B J, Goldsmith J C. The uncritical use of high-tech medical imaging. N Engl J Med 2010; 363: 4-6.
92. Gardner R L, Sarkar U, Maselli J H, Gonzales R. Factors associated with longer ED lengths of stay. Am J Emerg Med 2007; 25(6): 643-650.
93. Kocher K E, Meurer W J, Mallamothu B K. Impact of testing and treatment on emergency department length of stay using a national database. Ann Emerg Med 2011(abstract); 58(4) supp: S246-S247.
94. Williams N, Bello M. Perforation rate relates to delayed presentation in childhood acute appendicitis. J R Coll Surg 1998; 43: 101-102.
95. Bickell et al. How time affects the risk of rupture in appendicitis. J Am Coll Surgeons 2006;
202: 401-406.
96. Cuschieri J, Florence M, Flum D R, Jurkovich G J, Lin P, et al. Negative appendectomy and imaging accuracy in the Washington state surgical care and outcomes assessment program. Ann Surg 2008; 248(4): 557-562.
97. Raman S S, Asuagwu F C, Kadell B, cryer H, et al. Effect of CT on false positive diagnosis of appendicitis and perforation. N Eng J Med 2008 (letter); 358 (9): 972-973.
98. Raja A S, Wright C, Sodickson A D, Zane R D, Schiff G D, et al. Negative appendectomy rate in the era of CT: an 18 year perspective. Radiology 2010; 256(2): 460-465.

We claim:

1. A method for assessing appendicitis in a subject, which method comprises:
a) determining values of a plurality of biomarkers in a sample from a subject;
b) combining said values of said biomarkers using a mathematical algorithm to produce a numerical test score; and
c) comparing said test score to a reference value to assess appendicitis in said subject.

2. The method of claim 1, wherein the values of biomarkers are selected from the group consisting of amounts, concentrations and activities of the biomarkers.

3. The method of claim 1, wherein the biomarkers are selected from the group consisting of myeloid related protein 8/14 (MRP 8/14), C-reactive protein (CRP), hyaluronan (HA), matrix metalloproteinase-9 (MMP-9), serum amyloid A1 (SAA 1), serum amyloid A2 (SAA 2), α1-antitrypsin (A1AT), epidermal growth factor (EGF), endothelial-leukocyte adhesion molecule 1 (ELAM-1 or E-Selectin), granulocyte colony-stimulating factor (G-CSF or GCSF), glutathione s-transferase omega-1 (GSTO1), interleukin-6 (IL-6), interleukin-8 (IL-8), junction plakoglobin (JUP), Layilin, lectin, galactose binding, soluble 3 (Lgals3), malate dehydrogenase (MDH or MADH), matrix metalloproteinase-1 (MMP-1), neural cell adhesion molecule 1 (NCAM 1), nuclear factor NF-kappa-B p105 subunit (NFKB-1), plasminogen activator inhibitor-1 (PAI-1), Parkinson disease (autosomal recessive, early onset) 7 (Park-7), procalcitonin (PCT), metallopeptidase inhibitor 1 (TIMP-1), urokinase-plasminogen activator (UPA), vascular endothelial growth factor D (VEGF-D), white blood cell count (WBC), absolute neutrophil count (ANC) and percent neutrophil in WBC (% NEU).

4. The method of claim 1, wherein the values of at least 2, 3, 4, 5, 6, 7, 8, 9 or 10 biomarkers are determined and combined into a test score.

5. The method of claim 1, wherein the biomarkers are selected from the group consisting of myeloid related protein 8/14 (MRP 8/14), C-reactive protein (CRP), hyaluronan (HA), matrix metalloproteinase-9 (MMP-9), serum amyloid A1 (SAA 1), serum amyloid A2 (SAA 2), white blood cell count (WBC), absolute neutrophil count (ANC) and percent neutrophil in WBC (% NEU).

6. The method of any of claim 1, wherein the combination of the biomarkers does not include the combination of CRP and WBC, CRP and WBC, CRP and SAA, SAA and WBC, or CRP, SAA and WBC.

7. The method of claim 1, wherein the biomarkers are selected from the group consisting of ANC and CRP; ANC and HA; ANC and MMP-9; ANC and MRP 8/14; ANC and % NEU; ANC and SAA; ANC and WBC; CRP and HA; CRP and MMP-9; CRP and MRP 8/14; CRP and % NEU; CRP and SAA; CRP and WBC; HA and MMP-9; HA and MRP 8/14; HA and % NEU; HA and SAA; HA and WBC; MMP-9 and MRP 8/14; MMP-9 and % NEU; MMP-9 and SAA; MMP-9 and WBC; MRP 8/14 and % NEU; MRP 8/14 and SAA; MRP 8/14 and WBC; % NEU and SAA; % NEU and WBC; SAA and WBC; ANC, CRP and HA; ANC, CRP and MMP-9; ANC, CRP and MRP 8/14; ANC, CRP and % NEU; ANC, CRP and SAA; ANC, CRP and WBC; ANC, HA and MMP-9; ANC, HA and MRP 8/14; ANC, HA and % NEU; ANC, HA and SAA; ANC, HA and WBC; ANC, MMP-9 and MRP 8/14; ANC, MMP-9 and % NEU; ANC, MMP-9 and SAA; ANC, MMP-9 and WBC; ANC, MRP 8/14 and % NEU; ANC, MRP 8/14 and SAA; ANC, MRP 8/14 and WBC; ANC, % NEU and SAA; ANC, % NEU and WBC; ANC, SAA and WBC; CRP, HA and MMP-9; CRP, HA and MRP 8/14; CRP, HA and % NEU; CRP, HA and SAA; CRP, HA and WBC; CRP, MMP-9 and MRP 8/14; CRP, MMP-9 and % NEU; CRP, MMP-9 and SAA; CRP, MMP-9 and WBC; CRP, MRP 8/14 and % NEU; CRP, MRP 8/14 and SAA; CRP, MRP 8/14 and WBC; CRP, % NEU and SAA; CRP, % NEU and WBC; CRP, SAA and WBC; HA, MMP-9 and MRP 8/14; HA, MMP-9 and % NEU; HA, MMP-9 and SAA; HA, MMP-9 and WBC; HA, MRP 8/14 and % NEU; HA, MRP 8/14 and SAA; HA, MRP 8/14 and WBC; HA, % NEU and SAA; HA, % NEU and WBC; HA, SAA and WBC; MMP-9, MRP 8/14 and % NEU; MMP-9, MRP 8/14 and SAA; MMP-9, MRP 8/14 and WBC; MMP-9, % NEU and SAA; MMP-9, % NEU and WBC; MMP-9, SAA and WBC; MRP 8/14, % NEU and SAA; MRP 8/14, % NEU and WBC; MRP 8/14, SAA and WBC; % NEU, SAA and WB C.

8. The method of claim 1, wherein the sample is selected from the group consisting of a serum, a plasma and a blood sample.

9. The method of claim 1, wherein the sample is a clinical sample.

10. The method of claim 1, wherein the values of the biomarkers are determined by a format selected from the group consisting of an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, surface plasmon resonance (SPR), chemiluminescence assay, lateral flow immunoassay, u-capture assay, inhibition assay and avidity assay.

11. The method of claim 1, wherein the subject is a human.

12. The method of claim 1, wherein the mathematical algorithm is selected from the group consisting of Naïve Bayesian Classifiers (NBC), Fisher Linear Discriminants (FLD) and Logistic Regression (LR).

13. The method of claim 1, wherein the reference value is a threshold value or a reference range.

14. The method of claim 1, which further comprises a step of separating a subject into a group of having high risk for appendicitis or a group of having low risk for appendicitis before determining values of the biomarkers in a sample from the subject.

15. The method of claim 1, which is used for diagnosis, prognosis, stratification, risk assessment, or treatment monitoring of appendicitis in a subject.

16. A device or system for assessing appendicitis in a subject, which device or system comprises:
a) means for determining values of a plurality of biomarkers in a sample from a subject; and
b) a computer readable medium containing executable instructions that when executed combine said values of said biomarkers using a mathematical algorithm to produce a numerical test score.

17. The device or system of claim 16, which further comprises a computer readable medium containing executable instructions that when executed compare the test score to a reference value to assess appendicitis in a subject, and/or the reference value.

18. The device or system of claim 16, wherein the means for determining values of a plurality of biomarkers comprises binding reagents that specifically bind to the biomarkers.

19. A method for assessing appendicitis in a subject, which method comprises:
a) separating a subject into a group of having high risk for appendicitis or a group of having low risk for appendicitis;
b) determining values of a plurality of biomarkers in a sample from said subject; and
c) comparing said values of said biomarker to a reference value of the corresponding group to assess appendicitis in said subject.

20. The method of claim 19, wherein the subject is separated into a group of having high or low risk for appendicitis by assessing general inflammation level of the subject.

21. The method of claim 19, wherein the subject is separated into a group of having high or low risk for appendicitis by assessing a physical sign or symptom selected from the group consisting of duration of a symptom, duration of abdominal pain, diffuse abdominal pain, focal right lower quadrant (RLQ) abdominal pain, RLQ tenderness, pain with percussion, rebound tenderness, pain with cough, hop, heel tap, difficulty in walking, pain migrated right iliac fossa/RLQ, history of similar pain, anorexia, nausea, vomiting, temperature, Rovsing's sign, and rigidity or guarding, WBC, ANC and percent neutrophil in WBC.

22. The method of claim 19, wherein the subject is separated into a group of having high or low risk for appendicitis by assessing a physical sign or symptom selected from the group consisting of RLQ tenderness, rebound tenderness, pain migrated right iliac fossa/RLQ, vomiting, rigidity or guarding, and Rovsing's sign.

23. The method of claim 19, wherein 6 physical signs or symptoms are assessed and the subject is separated into a group of having high risk for appendicitis when at least 3 physical signs or symptoms are positive.

24. The method of claim 19, wherein the values of the biomarkers are selected from the group consisting of amount, concentration and activity of the biomarker.

25. The method of claim 19, wherein the biomarkers are selected from the group consisting of myeloid related protein 8/14 (MRP 8/14), C-reactive protein (CRP), hyaluronan (HA), matrix metalloproteinase-9 (MMP-9), serum amyloid A1 (SAA 1), serum amyloid A2 (SAA 2), α1-antitrypsin (A1AT), epidermal growth factor (EGF), endothelial-leukocyte adhesion molecule 1 (ELAM-1 or E-Selectin), granulocyte colony-stimulating factor (G-CSF or GCSF), glutathione s-transferase omega-1 (GSTO1), interleukin-6 (IL-6), interleukin-8 junction plakoglobin (JUP), Layilin, lectin, galactose binding, soluble 3 (Lgals3), malate dehydrogenase (MDH or MADH), matrix metalloproteinase-1 (MMP-1), neural cell adhesion molecule 1 (NCAM 1), nuclear factor NF-kappa-B p105 subunit (NFKB-1), plasminogen activator inhibitor-1 (PAI-1), Parkinson disease (autosomal recessive, early onset) 7 (Park-7), procalcitonin (PCT), metallopeptidase inhibitor 1 (TIMP-1), urokinase-plasminogen activator (UPA), vascular endothelial growth factor D (VEGF-D), white blood cell count (WBC), absolute neutrophil count (ANC) and percent neutrophil in WBC (% NEU).

26. The method of claim 19, wherein the biomarker are selected from the group consisting of myeloid related protein 8/14 (MRP 8/14), C-reactive protein (CRP), hyaluronan (HA), matrix metalloproteinase-9 (MMP-9), serum amyloid A1 (SAA 1), serum amyloid A2 (SAA 2), white blood cell count (WBC), absolute neutrophil count (ANC) and percent neutrophil in WBC.

27. The method of claim 19, wherein the values of at least 2, 3, 4, 5, 6, 7, 8, 9 or 10 biomarkers are determined and compared to the corresponding reference value(s).

28. The method of claim 19, wherein the sample is selected from the group consisting of a serum, a plasma and a blood sample.

29. The method of claim 19, wherein the sample is a clinical sample.

30. The method of claim 19, wherein the value of the biomarker is determined by a format selected from the group consisting of an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, surface plasmon resonance (SPR), chemiluminescence assay, lateral flow immunoassay, u-capture assay, inhibition assay and avidity assay.

31. The method of claim 19, wherein the subject is a human.

32. The method of claim 19, wherein the reference value is a threshold value or a reference range.

33. A kit or device for assessing appendicitis in a subject, which kit or device comprises:
a) means for assessing appendicitis risk in a subject; and
b) means for determining value of a biomarker in a sample from said subject.

34. The kit or device of claim 33, wherein the means for assessing appendicitis risk comprises means for assessing a physical sign or symptom selected from the group consisting of duration of a symptom, duration of abdominal pain, diffuse abdominal pain, focal right lower quadrant (RLQ) abdominal pain, RLQ tenderness, pain with percussion, rebound tenderness, pain with cough, hop, heel tap, difficulty in walking, pain migrated right iliac fossa/RLQ, history of similar pain, anorexia, nausea, vomiting, temperature, Rovsing's sign, and rigidity or guarding, WBC, ANC and percent neutrophil in WBC.

35. The kit or device of claim 33, wherein the means for determining value of a biomarker comprises a binding reagent that specifically binds to the biomarker.
